(19) United States
(12) Patent Application Publication
(10) Pub. No.: US 2018/0126784 A1
(43) Pub. Date: May 10, 2018

(54) RUBBER COMPOSITION

(71) Applicant: SUMITOMO CHEMICAL COMPANY, LIMITED, Tokyo (JP)

(72) Inventors: Orhan OZTURK, Tokyo (JP); Yasuo UEKITA, Tokyo (JP)

(73) Assignee: SUMITOMO CHEMICAL COMPANY, LIMITED, Tokyo (JP)

(21) Appl. No.: 15/570,040

(22) PCT Filed: Apr. 28, 2016
(86) PCT No.: PCT/JP2016/063317

§ 371 Date: Oct. 27, 2017

(30) Foreign Application Priority Data
Apr. 30, 2015 (JP) 2015-093469

(51) Int. Cl. B60C 1/00; B60C 11/00; B60C 5/14; C07C 233/44; C08K 5/00; C08K 5/20; C08K 5/10; C08K 3/04; C08L 21/00; C08L 9/00

(52) U.S. Cl. CPC B60C 1/00; B60C 11/00; B60C 5/14; C07C 233/44; C08L 9/00; C08K 5/20; C08K 5/10; C08K 3/04; C08L 21/00; C08K 5/0025

(57) ABSTRACT

The present invention provides a rubber composition obtained by kneading a compound represented by the formula (I), a rubber component and a filler, and the like:

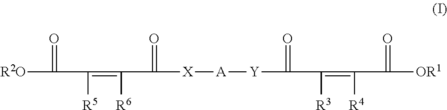

wherein the groups in the formula (I) are as described in the DESCRIPTION.

### TECHNICAL FIELD

The present invention relates to a rubber composition and the like.

### BACKGROUND ART

In recent years, improvement of fuel consumption (namely, low fuel consumption) of automobiles is requested from the demand of environmental protection. In the field of automotive tires, it is known that reduction of the loss factor (tan δ) possessed by vulcanized rubber compositions used for tire production improves fuel consumption of automobiles (non-patent document 1).

### DOCUMENT LIST

### Non-Patent Document

non-patent document 1: “GOMU GIJYUTU NYUUMON (Introduction to Rubber Technology)” edited by The Society of Rubber Science and Technology, Japan, Maruzen Company, Limited, pp. 123-124

### SUMMARY OF THE INVENTION

### Problems to be Solved by the Invention

The present invention aims to lower the loss factor (tan δ) of vulcanized rubber compositions.

### Means of Solving the Problems

The present invention capable of achieving the above-mentioned object is as described below.

[1] A rubber composition obtained by kneading a compound represented by the formula (I):

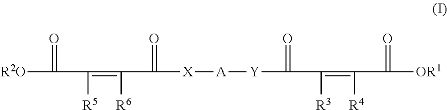

[in the formula (I),

R¹ and R² are each independently a C₁₋₁₂ alkyl group optionally having one or more substituents or a C₃₋₁₀ cycloalkyl group optionally having one or more substituents,

R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents,

X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and

A is a divalent C₆₋₁₂ aromatic hydrocarbon group optionally having one or more substituents],

a rubber component and a filler.
[2] A rubber composition comprising a compound represented by the formula (I):

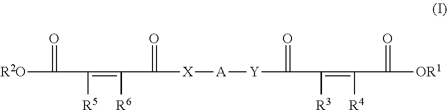

[in the formula (I),

R¹ and R² are each independently a C₁₋₁₂ alkyl group optionally having one or more substituents or a C₃₋₁₀ cycloalkyl group optionally having one or more substituents,

R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents,

X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and

A is a divalent C₆₋₁₂ aromatic hydrocarbon group optionally having one or more substituents],

a rubber component and a filler.
[3] The rubber composition of the aforementioned [1] or [2], wherein R³, R⁴, R⁵ and R⁶ are each a hydrogen atom.
[4] The rubber composition of the aforementioned [1] or [2], wherein the compound represented by the formula (I) is a compound represented by the formula (II):

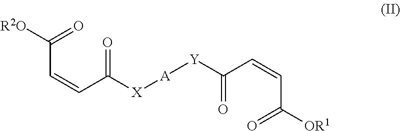

[in the formula (II),

R¹ and R² are each independently a C₁₋₁₂ alkyl group optionally having one or more substituents or a C₃₋₁₀ cycloalkyl group optionally having one or more substituents,

X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and

A is a divalent C₆₋₁₂ aromatic hydrocarbon group optionally having one or more substituents].

[5] The rubber composition of any one of the aforementioned [1]-[4], wherein R¹ and R² are each independently a linear C₁₋₆ alkyl group.
[6] The rubber composition of any one of the aforementioned [1]-[5], wherein A is a phenylene group.
[7] The rubber composition of any one of the aforementioned [1]-[6], wherein X and Y are each independently —NH— or —O—.
[8] The rubber composition of any one of the aforementioned [1]-[6], wherein X and Y are each —NH—.
[9] The rubber composition of any one of the aforementioned [1]-[8], wherein the rubber component comprises a diene rubber.
[10] The rubber composition of any one of the aforementioned [1]-[9], wherein the filler comprises carbon black.
[11] The rubber composition of any one of the aforementioned [1]-[10], which is obtained by further kneading a sulfur component.
[12] The rubber composition of any one of the aforementioned [1]-[10], further comprising a sulfur component.
[13] A vulcanized rubber composition obtained by vulcanizing the rubber composition of the aforementioned [11] or [12].
[14] A vulcanized tire produced using the rubber composition of the aforementioned [11] or [12].
[15] A vulcanized tire comprising the vulcanized rubber composition of the aforementioned [13].
[16] A tire belt member comprising the vulcanized rubber composition of the aforementioned [13] and a steel cord.
[17] A tire carcass member comprising the vulcanized rubber composition of the aforementioned [13] and a carcass fiber cord.
[18] A tire member comprising the vulcanized rubber composition of the aforementioned [13].
[19] The tire member of the aforementioned [18], which is a tire side wall member, a tire inner liner member, a tire cap tread member or a tire under tread member.
[20] A loss factor lowering agent comprising a compound represented by the formula (I):

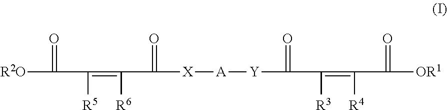

[in the formula (I),

R¹ and R² are each independently a C₁₋₁₂ alkyl group optionally having one or more substituents or a C₃₋₁₀ cycloalkyl group optionally having one or more substituents,

R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents,

X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and

A is a divalent C₆₋₁₂ aromatic hydrocarbon group optionally having one or more substituents],

which is for a vulcanized rubber composition.
[21] The loss factor lowering agent of the aforementioned [20], wherein R³, R⁴, R⁵ and R⁶ are each a hydrogen atom.
[22] The loss factor lowering agent of the aforementioned [20], wherein the compound represented by the formula (I) is a compound represented by the formula (II):

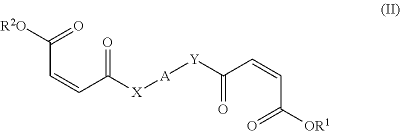

[in the formula (II),

R¹ and R² are each independently a C₁₋₁₂ alkyl group optionally having one or more substituents or a C₃₋₁₀ cycloalkyl group optionally having one or more substituents,

X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and

A is a divalent C₆₋₁₂ aromatic hydrocarbon group optionally having one or more substituents].

[23] The loss factor lowering agent of any one of the aforementioned [20]-[22], wherein R¹ and R² are each independently a linear C₁₋₆ alkyl group.
[24] The loss factor lowering agent of any one of the aforementioned [20]-[23], wherein A is a phenylene group.
[25] The loss factor lowering agent of any one of the aforementioned [20]-[24], wherein X and Y are each independently —NH— or —O—.
[26] The loss factor lowering agent of any one of the aforementioned [20]-[24], wherein X and Y are each —NH—.
[27] Use of a compound represented by the formula (I):

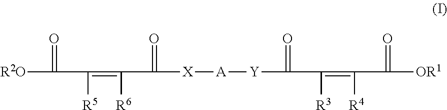

[in the formula (I),

R¹ and R² are each independently a C₁₋₁₂ alkyl group optionally having one or more substituents or a C₃₋₁₀ cycloalkyl group optionally having one or more substituents,

R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents,

X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and

A is a divalent C₆₋₁₂ aromatic hydrocarbon group optionally having one or more substituents], for lowering a loss factor of a vulcanized rubber composition.

[28] The use of the aforementioned [27], wherein R³, R⁴, R⁵ and R⁶ are each a hydrogen atom.
[29] The use of the aforementioned [27], wherein the compound represented by the formula (I) is a compound represented by the formula (II):

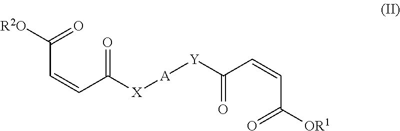

[in the formula (II),

R¹ and R² are each independently a C₁₋₁₂ alkyl group optionally having one or more substituents or a C₃₋₁₀ cycloalkyl group optionally having one or more substituents,

X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and

A is a divalent C₆₋₁₂ aromatic hydrocarbon group optionally having one or more substituents].

[30] The use of any one of the aforementioned [27]-[29], wherein R¹ and R² are each independently a linear C₁₋₆ alkyl group.
[31] The use of any one of the aforementioned [27]-[30], wherein A is a phenylene group.
[32] The use of any one of the aforementioned [27]-[31], wherein X and Y are each independently —NH— or —O—.
[33] The use of any one of the aforementioned [27]-[31], wherein X and Y are each —NH—.
[34] A method of lowering a loss factor of a vulcanized rubber composition, comprising kneading a compound represented by the formula (I):

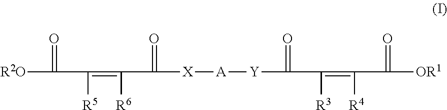

[in the formula (I),

R¹ and R² are each independently a C₁₋₁₂ alkyl group optionally having one or more substituents or a C₃₋₁₀ cycloalkyl group optionally having one or more substituents,

R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents,

X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and

A is a divalent C₆₋₁₂ aromatic hydrocarbon group optionally having one or more substituents], a rubber component, a filler, and a sulfur component.

[35] The method of the aforementioned [34], wherein R³, R⁴, R⁵ and R⁶ are each a hydrogen atom.
[36] The method of the aforementioned [34], wherein the compound represented by the formula (I) is a compound represented by the formula (II):

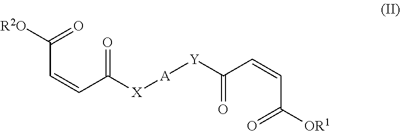

[in the formula (II),

R¹ and R² are each independently a C₁₋₁₂ alkyl group optionally having one or more substituents or a C₃₋₁₀ cycloalkyl group optionally having one or more substituents,

X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and

A is a divalent C₆₋₁₂ aromatic hydrocarbon group optionally having one or more substituents].

[37] The method of any one of the aforementioned [34]-[36], wherein R¹ and R² are each independently a linear C₁₋₆ alkyl group.
[38] The method of any one of the aforementioned [34]-[37], wherein A is a phenylene group.
[39] The method of any one of the aforementioned [34]-[38], wherein X and Y are each independently —NH— or —O—.
[40] The method of any one of the aforementioned [34]-[38], wherein X and Y are each —NH—.
[41] The method of any one of the aforementioned [34]-[40], wherein the rubber component comprises a diene rubber.
[42] The method of any one of the aforementioned [34]-[41], wherein the filler comprises carbon black.

### Effect of the Invention

According to the present invention, a loss factor of a vulcanized rubber composition can be lowered.

### DESCRIPTION OF EMBODIMENTS

### <Compound Represented by the Formula (I)>

To lower a loss factor (tan δ) of a vulcanized rubber composition, the present invention uses a compound represented by the formula (I):

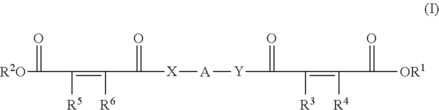

In the following, “a compound represented by the formula (I)” and the like are sometimes abbreviated as “compound (I)” and the like. The present invention provides (i) a rubber composition obtained by kneading compound (I) and a rubber component and a filler (rubber composition containing compound (I), a rubber component and a filler), (ii) a rubber composition obtained by kneading compound (I) and a rubber component and a filler and a sulfur component (rubber composition containing compound (I), a rubber component, a filler and a sulfur component), (iii) a vulcanized rubber composition obtained by vulcanizing the rubber composition of the aforementioned (ii) and a product obtained therefrom (vulcanized tire, tire belt member, tire carcass member, other tire member), (iv) a loss factor lowering agent containing compound (I), for a vulcanized rubber composition, (v) use of compound (I) for lowering a loss factor of a vulcanized rubber composition, and (vi) a method of lowering a loss factor of a vulcanized rubber composition, including kneading compound (I) and a rubber component and a filler and a sulfur component. In the present specification, the “loss factor lowering agent for a vulcanized rubber composition” means an agent used for lowering a loss factor (tan δ) of a vulcanized rubber composition.

Compound (I) may react with a rubber component and/or a filler (particularly, carbon black) during kneading to form a compound different from compound (I). However, it is practically impossible for the current technique for analyzing solid rubber compositions to directly specify the aforementioned compound possibly formed in the rubber composition by the structure or property thereof. For multiphasic protection of the present invention, therefore, the rubber composition of the present invention is specified as “a rubber composition containing compound (I), a rubber component and a filler” and “a rubber composition obtained by kneading compound (I) and a rubber component and a filler” in the present specification and claims. In the following, compound (I) is first explained.

R¹ and R² are each independently a C₁₋₁₂ alkyl group optionally having one or more substituents or a C₃₋₁₀ cycloalkyl group optionally having one or more substituents. R¹ and R² are preferably the same group.

In the present specification, “C_{x-y}” means that the carbon atom number is not less than x and not more than y (x, y: an integer).

In the present specification, the alkyl group includes both linear alkyl group and branched-chain alkyl group. In the present specification, examples of the “C₁₋₁₂ alkyl group” include methyl group, ethyl group, propyl group, butyl group, pentyl group, hexyl group, heptyl group, octyl group, nonyl group, decyl group, undecyl group, dodecyl group, isopropyl group, s-butyl group, t-butyl group, 2-methylbutyl group, 2-ethylbutyl group, 2-propylbutyl group, 3-methylbutyl group, 3-ethylbutyl group, 3-propylbutyl group, 2-methylpentyl group, 2-ethylpentyl group, 2-propylpentyl group, 3-methylpentyl group, 3-ethylpentyl group, 3-propylpentyl group, 4-methylpentyl group, 4-ethylpentyl group, 4-propylpentyl group, 2-methylhexyl group, 2-ethylhexyl group, 2-propylhexyl group, 3-methylhexyl group, 3-ethylhexyl group, 3-propylhexyl group, 4-methylhexyl group, 4-ethylhexyl group, 4-propylhexyl group, 5-methylhexyl group, 5-ethylhexyl group, 5-propylhexyl group. In the present specification, examples of the “C₁₋₆ alkyl group” include those in the aforementioned “C₁₋₁₂ alkyl group” which have a carbon number of 1-6.

In the present specification, examples of the “C₃₋₁₀ cycloalkyl group” include cyclopropyl group, cyclobutyl group, cyclopentyl group, cyclohexyl group, cycloheptyl group, cyclooctyl group, cyclononyl group, cyclodecyl group. In the present specification, examples of the “C₃₋₆ cycloalkyl group” include those in the aforementioned “C₃₋₁₀ cycloalkyl group” which have a carbon number of 3-6.

Examples of the substituent that the C₁₋₁₂ alkyl group optionally has include C₁₋₆ alkoxy group, C₁₋₆ alkoxy-carbonyl group, C₁₋₇ acyl group, C₁₋₇ acyl-oxy group, and C₆₋₁₄ aryl group optionally having one or more substituents.

Examples of the substituent that the C₃₋₁₀ cycloalkyl group optionally has include C₁₋₆ alkyl group, C₁₋₆ alkoxy group, C₁₋₆ alkoxy-carbonyl group, C₁₋₇ acyl group, C₁₋₇ acyl-oxy group, and C₆₋₁₄ aryl group optionally having one or more substituents.

In the present specification, the alkoxy group encompasses both linear alkoxy group and branched-chain alkoxy group. In the present specification, examples of the “C₁₋₆ alkoxy group” include methoxy group, ethoxy group, propoxy group, isopropoxy group, butoxy group, isobutoxy group, sec-butoxy group, tert-butoxy group, pentyloxy group, and hexyloxy group.

In the present specification, examples of the “C₁₋₇ acyl group” include formyl group, C₁₋₆ alkyl-carbonyl group (e.g., acetyl group, pivaloyl group), and benzoyl group.

In the present specification, examples of the “C₆₋₁₄ aryl group” include phenyl group, 1-naphthyl group, 2-naphthyl group, 1-anthryl group, 2-anthryl group, and 9-anthryl group.

Examples of the substituent that the C₆₋₁₄ aryl group optionally has include C₁₋₆ alkyl group, C₁₋₆ alkoxy group, C₁₋₆ alkoxy-carbonyl group, C₁₋₇ acyl group, and C₁₋₇ acyl-oxy group. In the present specification, examples of the “C₆₋₁₄ aryl group having one or more substituents” include tolyl group, and xylyl group.

In the present specification, examples of the “C₁₋₆ alkoxy group” included in the C₁₋₆ alkoxy-carbonyl group and the “C₁₋₇ acyl group” included in the C₁₋₇ acyl-oxy group include those mentioned above.

R¹ and R² are each independently and preferably a C₁₋₁₂ alkyl group, more preferably a C₁₋₆ alkyl group, further preferably a linear C₁₋₆ alkyl group (i.e., methyl group, ethyl group, propyl group, butyl group, pentyl group, hexyl group).

R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents. R³ and R⁶ are preferably the same group. In addition, R⁴ and R⁵ are preferably the same group.

In the present specification, as the “halogen atom”, fluorine, chlorine, bromine and iodine can be mentioned.

Examples of the substituent that the C₁₋₆ alkoxy group optionally has include C₁₋₆ alkyl group, C₁₋₆ alkoxy group, C₁₋₆ alkoxy-carbonyl group, C₁₋₇ acyl group, C₁₋₇ acyl-oxy group, and C₆₋₁₄ aryl group optionally having one or more substituents.

Examples of the substituent that the C₁₋₆ alkyl group optionally has include C₁₋₆ alkoxy group, C₁₋₆ alkoxy-carbonyl group, C₁₋₇ acyl group, C₁₋₇ acyl-oxy group, and C₆₋₁₄ aryl group optionally having one or more substituents.

Examples of the substituent that the C₃₋₆ cycloalkyl group optionally has include C₁₋₆ alkyl group, C₁₋₆ alkoxy group, C₁₋₆ alkoxy-carbonyl group, C₁₋₇ acyl group, C₁₋₇ acyl-oxy group, and C₆₋₁₄ aryl group optionally having one or more substituents.

Examples of the “C₃₋₁₀ cycloalkenediyl group formed by R³ and R⁴, which are bonded to each other, together with a carbon atom bonded thereto” and the “C₃₋₁₀ cycloalkenediyl group formed by R⁵ and R⁶, which are bonded to each other, together with a carbon atom bonded thereto” include cyclopropene-1,2-diyl group, cyclobutene-1,2-diyl group, cyclopentene-1,2-diyl group, cyclohexene-1,2-diyl group, cycloheptene-1,2-diyl group, cyclooctene-1,2-diyl group, cyclononene-1,2-diyl group, and cyclodecene-1,2-diyl group.

Examples of the substituent that the C₃₋₁₀ cycloalkenediyl group optionally has include C₁₋₆ alkyl group, C₁₋₆ alkoxy group, C₁₋₆ alkoxy-carbonyl group, C₁₋₇ acyl group, C₁₋₇ acyl-oxy group, and C₆₋₁₄ aryl group optionally having one or more substituents.

R³, R⁴, R⁵ and R⁶ are each independently preferably a hydrogen atom, a hydroxy group, or a C₁₋₆ alkyl group, more preferably, a hydrogen atom or a hydroxy group. R³, R⁴, R⁵ and R¹ are each further preferably a hydrogen atom.

When R³ and R⁴ are not bonded, R³ and R⁴ may be on the same side of a double bond, or on the opposite side. When R⁵ and R⁶ are not bonded, R⁵ and R⁶ may be on the same side of a double bond, or on the opposite side. When R³ and R⁴ are not bonded and R⁵ and R⁶ are not bonded, R³ and R⁴ are preferably on the same side of a double bond, and R⁵ and R⁶ are preferably on the same side of a double bond.

Of compound (I), a compound represented by the following formula (II) wherein R³, R⁴, R⁵ and R⁶ are each a hydrogen atom, R³ and R⁴ are on the same side of a double bond, and R⁵ and R⁶ are on the same side of a double bond is preferable.

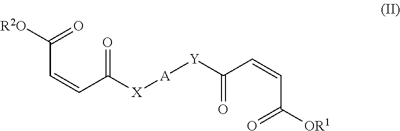

wherein X and Y are each independently —NR⁷—, —O—, —S—, or a C₁₋₁₂ alkanediyl group optionally having one or more substituents, and R⁷ is a hydrogen atom or a C₁₋₁₂ alkyl group optionally having one or more substituents. X and Y are preferably the same group.

In the present specification, the alkanediyl group encompasses both linear alkanediyl group and branched-chain alkanediyl group. In the present specification, examples of the “C₁₋₁₂ alkanediyl group” include methylene group, ethylene group, trimethylene group, tetramethylene group, pentamethylene group, hexamethylene group, propylene group, 1-methyltrimethylene group, 2-methyltrimethylene group, 1-ethyltrimethylene group, 2-ethyltrimethylene group, 1-propyltrimethylene group, 2-propyltrimethylene group, 1-methyltetramethylene group, 2-methyltetramethylene group, 1-ethyltetramethylene group, 2-ethyltetramethylene group, 1-propyltetramethylene group, 2-propyltetramethylene group, 1-methylpentamethylene group, 2-methylpentamethylene group, 3-methylpentamethylene group, 1-ethylpentamethylene group, 2-ethylpentamethylene group, 3-ethylpentamethylene group, 1-propylpentamethylene group, 2-propylpentamethylene group, 3-propylpentamethylene group, 1-methylhexamethylene group, 2-methylhexamethylene group, 3-methylhexamethylene group, 1-ethylhexamethylene group, 2-ethylhexamethylene group, 3-ethylhexamethylene group, 1-propylhexamethylene group, 2-propylhexamethylene group, and 3-propylhexamethylene group.

Examples of the substituent that the C₁₋₁₂ alkanediyl group optionally has include C₁₋₆ alkoxy group, C₁₋₆ alkoxy-carbonyl group, C₁₋₇ acyl group, C₁₋₇ acyl-oxy group, C₆₋₁₄ aryl group optionally having one or more substituents.

X and Y are each independently preferably —NR⁷— (in the aforementioned formula, R⁷ is a hydrogen atom or C₁₋₁₂ alkyl group), —O— or —S—, more preferably —NR⁷— (in the aforementioned formula, R⁷ is a hydrogen atom or a C₁₋₁₂ alkyl group) or —O—, further preferably —NH— or —O—. X and Y are each particularly preferably —NH—.

A is a divalent C₆₋₁₂ aromatic hydrocarbon group optionally having one or more substituents.

In the present specification, examples of the “divalent C₆₋₁₂ aromatic hydrocarbon” include phenylene group (e.g., 1,4-phenylene group), naphthylene group (e.g., 1,4-naphthylene group, 1,5-naphthylene group, 2,6-naphthylene group, 2,7-naphthylene group), and biphenyldiyl group (e.g., 1,1′-biphenyl-4,4′-diyl group).

Examples of the substituent that the divalent C₆₋₁₂ aromatic hydrocarbon group optionally has include C₁₋₆ alkyl group, C₁₋₆ alkoxy group, C₁₋₆ alkoxy-carbonyl group, C₁₋₇ acyl group, and C₁₋₇ acyl-oxy group.

A is preferably a phenylene group optionally having one or more substituents or a naphthylene group optionally having one or more substituents, more preferably a phenylene group optionally having one or more substituents, further preferably a phenylene group, particularly preferably a 1,4-phenylene group.

While specific examples of compound (I) are shown below, the scope of the present invention is not limited thereto.

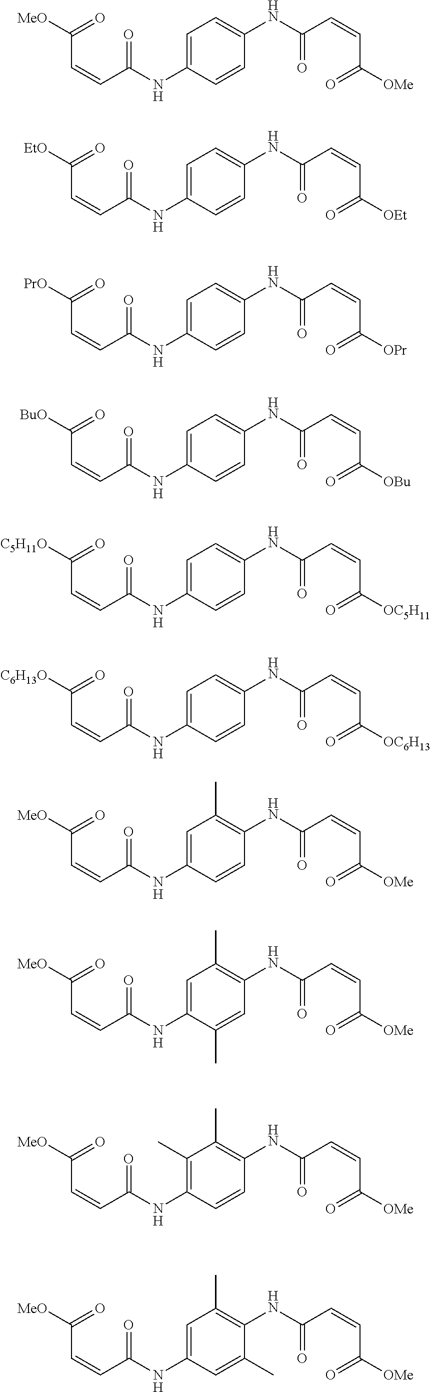

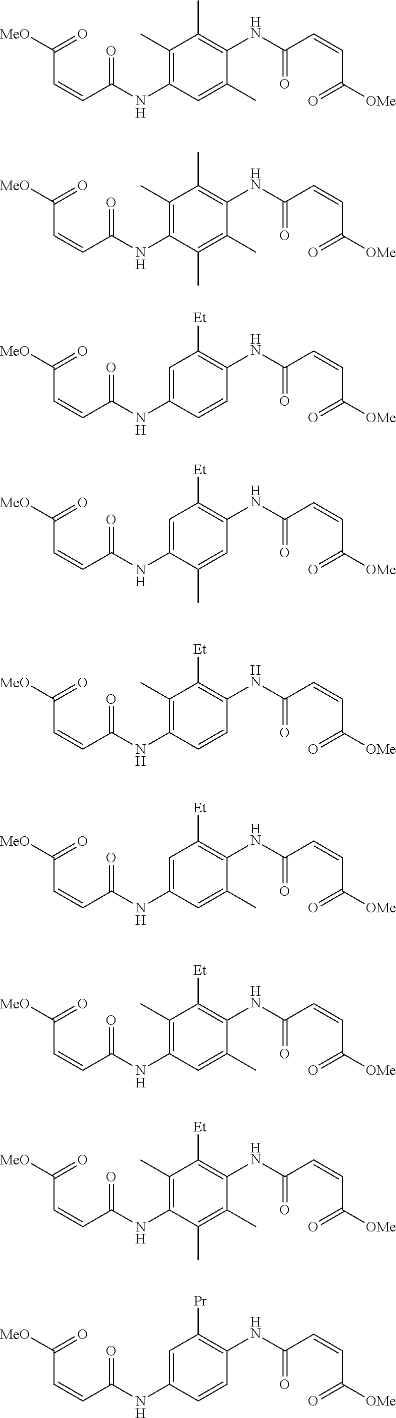

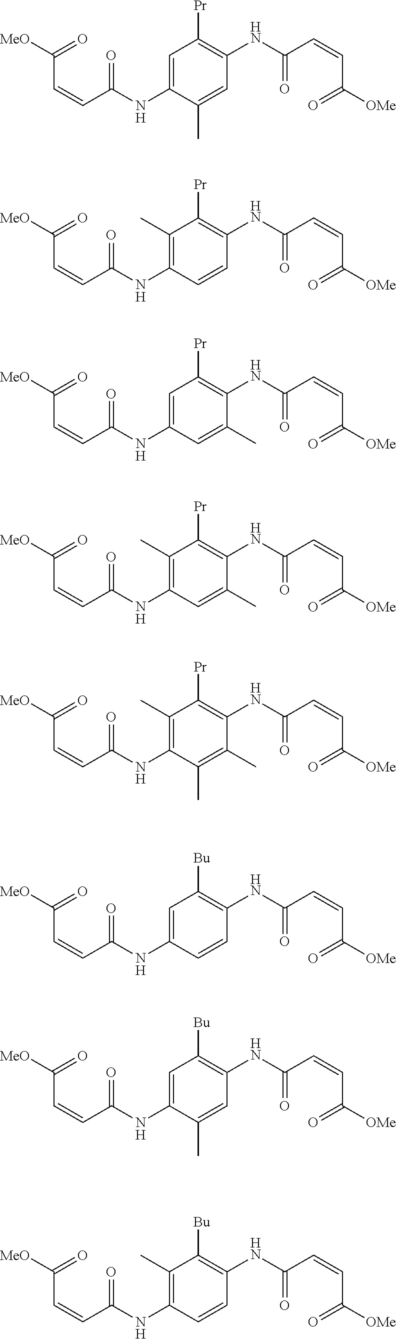

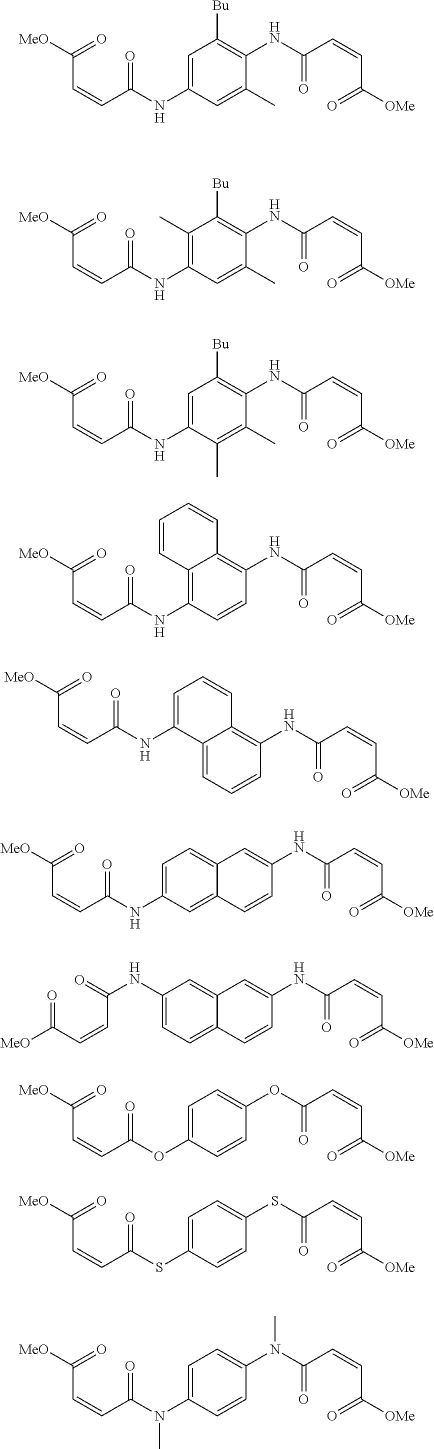

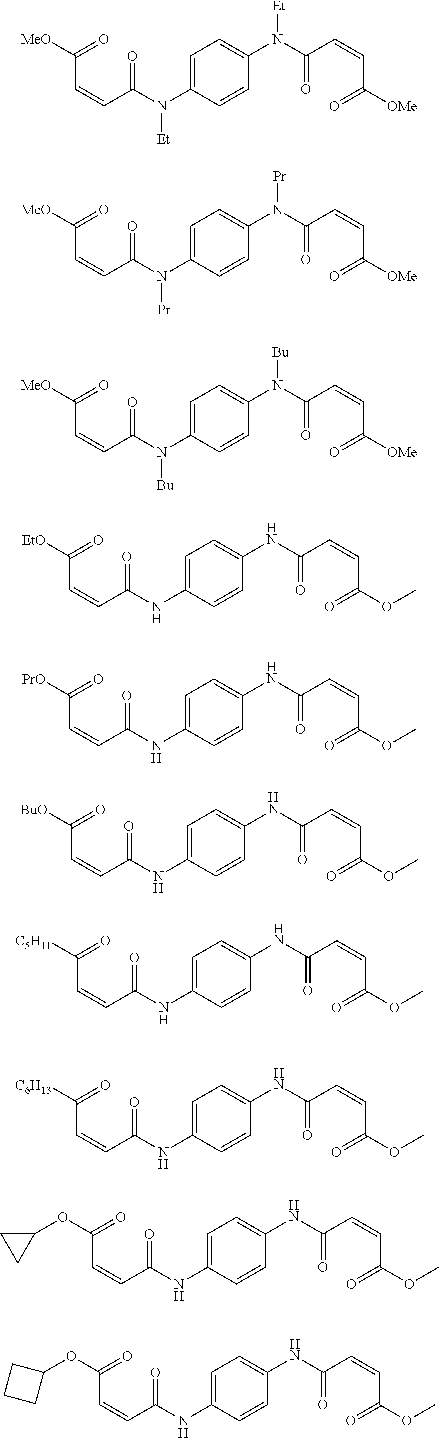

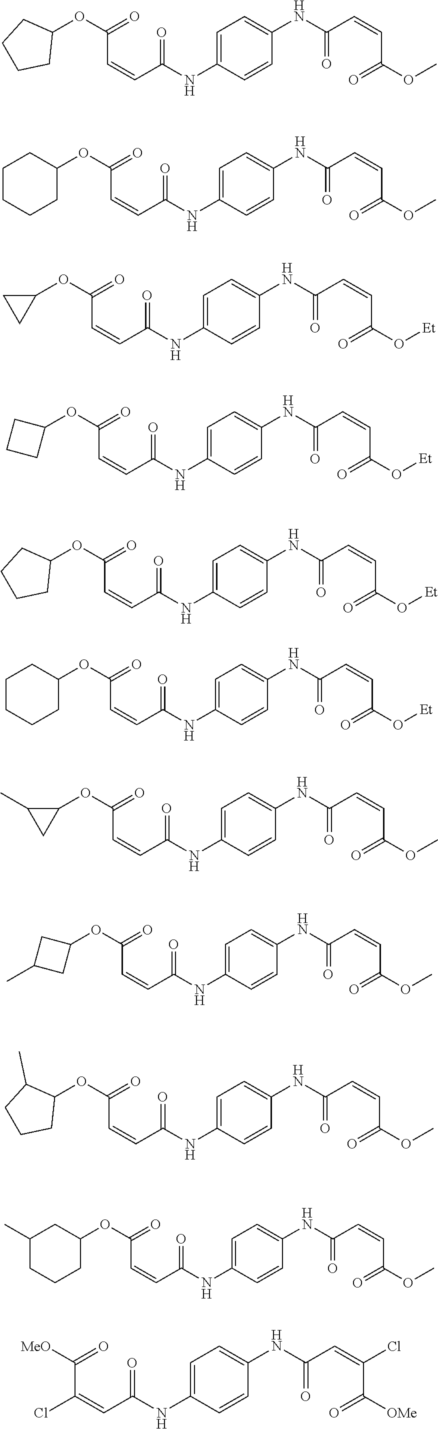

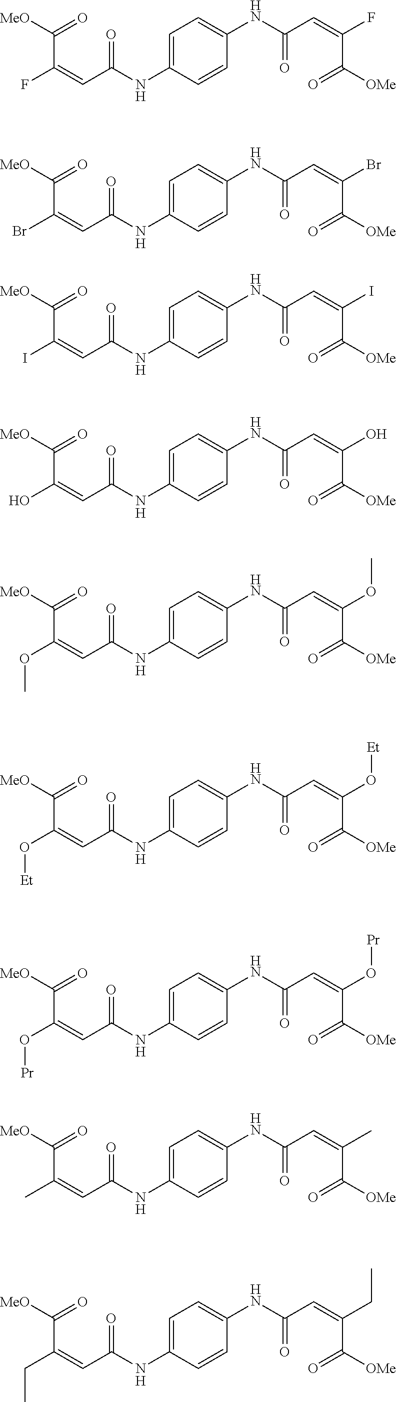

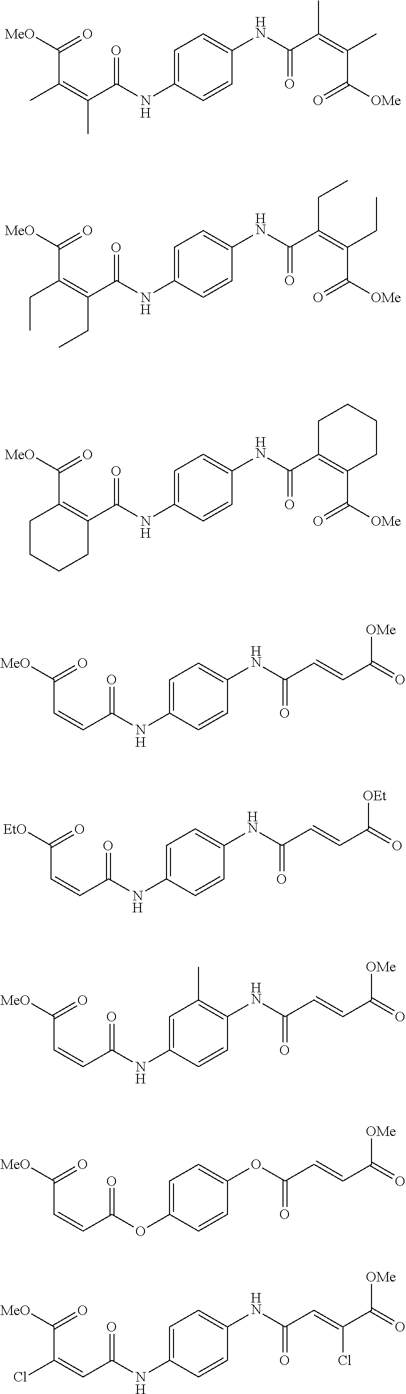

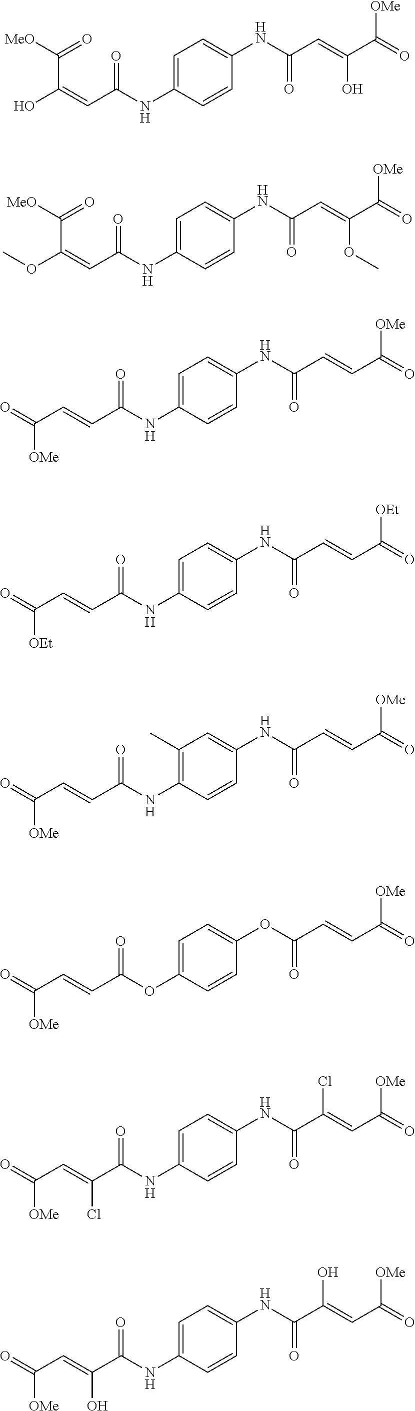

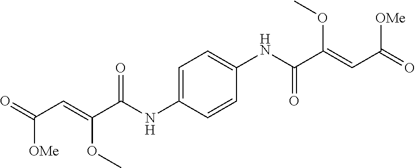 [SMILES: COC(=O)/C=C(\OC)C(=O)Nc1ccc(NC(=O)/C(=C/C(=O)OC)OC)cc1]

For example, compound (I) can be produced as shown in the following formula (the definitions of the symbols in the following formulas are as defined above).

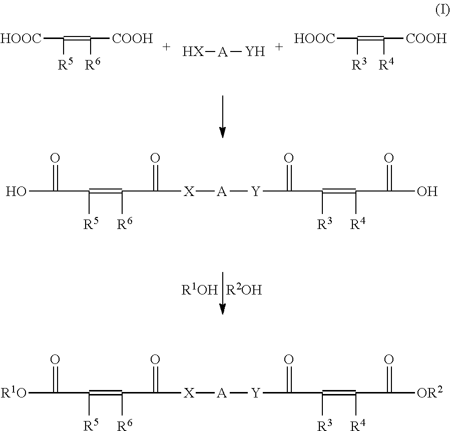

In the synthesis of compound (I), a known protecting group may be used. Protecting groups can be introduced and removed by a known method, for example, the method described in Greene's PROTECTIVE GROUPS IN ORGANIC SYNTHESIS, 4th Edition, JOHN WILLY&SONS (2006) and the like.

Compound (I) may be a solvate. The solvate of compound (I) can be produced by, for example, recrystallizing compound (I) from a solvent (e.g., water or methanol). Examples of the solvate of compound (I) include hydrate and methanol solvate.

The amount of compound (I) is preferably 0.01-20 parts by weight, more preferably 0.05-15 parts by weight, further preferably 0.05-10 parts by weight, per 100 parts by weight of the rubber component.

### <Rubber Component>

The rubber component used in the present invention is explained below. Examples of the rubber component include natural rubber (NR) and modified natural rubbers (e.g., epoxydized natural rubber, deproteinized natural rubber); various synthetic rubbers such as polyisoprene rubber (IR), styrene-butadiene copolymer rubber (SBR), polybutadiene rubber (BR), acrylonitrile-butadiene copolymer rubber (NBR), isoprene-isobutylene copolymer rubber (IIR), ethylene-propylene-diene copolymer rubber (EPDM), halogenated butyl rubber (HR) and the like. Only one kind of rubber component may be used, or two or more kinds thereof may be used in combination.

The rubber component preferably includes a diene rubber. Examples of the diene rubber include natural rubber, modified natural rubber, polyisoprene rubber, chloroprene rubber, styrene-butadiene copolymer rubber, polybutadiene rubber, nitrile rubber and the like. The diene rubber is preferably highly unsaturated, more preferably natural rubber. It is also effective to use natural rubber and other rubber (e.g., styrene-butadiene copolymer rubber, polybutadiene rubber) in combination. When the diene rubber (particularly, natural rubber) is used, the amount of the diene rubber in the rubber component is preferably 50-100 wt %, more preferably 80-100 wt %.

Examples of the natural rubber include natural rubber with grades such as RSS #1, RSS #3, TSR20, SIR20 and the like. Examples of the epoxydized natural rubber include those with a degree of epoxidization of 10-60 mol % (e.g., ENR25 and ENR50 manufactured by Kumpulan Guthrie). As the deproteinized natural rubber, deproteinized natural rubber having a total nitrogen content of not more than 0.3 wt % is preferable. Examples of the other modified natural rubber include modified natural rubber containing a polar group obtained by reacting natural rubber with 4-vinylpyridine, N,N,-dialkylaminoethyl acrylate (e.g., N,N,-diethylaminoethyl acrylate), 2-hydroxy acrylate and the like.

Examples of the SBR include emulsion-polymerized SBR and solution-polymerized SBR described in “GOMU KOGYO BINRAN (Rubber Industry Handbook)<fourth edition>” pp. 210-211 edited by The Society of Rubber Science and Technology, Japan. Of these, solution-polymerized SBR is preferable for a rubber composition for a tread.

Examples of the solution-polymerized SBR include modified solution-polymerized SBR having at least one element of nitrogen, tin and silicon on the molecular terminal, which is obtained by modifying with a modifying agent. Examples of the modifying agent include lactam compound, amide compound, urea compound, N,N-dialkylacrylamide compound, isocyanate compound, imide compound, silane compound having an alkoxy group, aminosilane compound, combined modifying agent of tin compound and silane compound having an alkoxy group, combined modifying agent of alkylacrylamide compound and silane compound having an alkoxy group, and the like. Specific examples of the modified solution-polymerized SBR include solution-polymerized SBR having a molecular terminal modified with 4,4′-bis(dialkylamino)benzophenone such as “Nipol (registered trade mark) NS116” manufactured by Zeon Corporation, solution-polymerized SBR having a molecular terminal modified with a halogenated tin compound such as “SL574” manufactured by JSR, silane modified solution-polymerized SBR such as “E10” and “E15” manufactured by Asahi Kasei Corporation, and the like.

In addition, oil-extended SBR obtained by adding oil such as process oil, aroma oil and the like to emulsion-polymerized SBR and solution-polymerized SBR are also preferable for a rubber composition for a tread.

As BR, any of solution-polymerized BR having a low vinyl content and solution-polymerized BR having a high vinyl content may be used, and solution-polymerized BR having a high vinyl content is preferable. A modified solution-polymerized BR having at least one element of nitrogen, tin and silicon on the molecular terminal, which is obtained by modifying with a modifying agent is particularly preferable. Examples of the modifying agent include 4,4′-bis(dialkylamino)benzophenone, halogenated tin compound, lactam compound, amide compound, urea compound, N,N-dialkylacrylamide compound, isocyanate compound, imide compound, silane compound having an alkoxy group (e.g., trialkoxysilane compound), aminosilane compound, combined modifying agent of tin compound and silane compound having an alkoxy group, combined modifying agent of alkylacrylamide compound and silane compound having an alkoxy group, and the like. Examples of the modified solution-polymerized BR include tin-modified BR such as “Nipol (registered trade mark) BR 1250H” manufactured by Zeon Corporation and the like.

BR can be preferably used for a rubber composition for a tread, and a rubber composition for a side wall. BR may be used as a blend with SBR and/or natural rubber (NR). In a rubber composition for a tread, for example, the amount of SBR and/or NR is 60-100 wt %, and the amount of BR is 0-40 wt %, in the rubber component. In a rubber composition for a side wall, preferably the amount of SBR and/or NR is 10-70 wt %, and the amount of BR is 90-30 wt %, more preferably, the amount of NR is 40-60 wt %, and the amount of BR is 60-40 wt %, in the rubber component. A blend of modified SBR and unmodified SBR, and a blend of modified BR and unmodified BR is and the like can be preferably used to a rubber composition for a tread and a rubber composition for a side wall.

When the rubber composition of the present invention is used for a tire tread, for example, SBR superior in the abrasion resistance and hysteresis loss reduction performance as a rubber component is used as a base material of passenger car tires, and NR having high strength is used as a base material optionally together with SBR in truck-bus tires, and it is preferable to used a blend of these base materials and BR where necessary, since a tread superior in abrasion resistance, fatigue resistance, and rebound resilience can be obtained.

When the rubber composition of the present invention is used for tire side walls, a blend of NR and SBR, or a blend of NR and BR is preferably used for passenger car tires, and a blend of NR and BR is preferably used for truck-bus tires, since bending resistance, and resistance to crack growth can be obtained.

When the rubber composition of the present invention is used for tire belts, NR and/or IR are/is preferably used as a rubber component, since high elastic modulus and good adhesiveness to reinforcing fibers can be obtained.

When the rubber composition of the present invention is used for tire inner liners, a blend of IIR, SBR and NR or a blend of IIR and NR is preferably used as a rubber component, since low gas permeability and bending resistance can be obtained.

As a diene rubber in the rubber composition, for example, the modified diene-based polymer described in WO 2012/057308, or the conjugated diene-based polymer described in JP-A-2012-140595 may be used.

### <Filler>

Examples of the filler include carbon black, silica (e.g., hydrated silica), aluminum hydroxide, ground bituminous coal, talc, clay (particularly, calcined clay), titanium oxide and the like. Of these, carbon black, silica, aluminum hydroxide and ground bituminous coal are preferable, carbon black and silica are more preferable, and carbon black is further preferable. When carbon black is used, the amount of carbon black in the filler is preferably 50-100 wt %, more preferably 70-100 wt %, further preferably 80-100 wt %.

Examples of carbon black include those described in “GOMU KOGYO BINRAN (Rubber Industry Handbook)<fourth edition>”, p. 494, edited by The Society of Rubber Science and Technology, Japan. Only one kind of carbon black may be used, or two or more kinds thereof may be used in combination. As carbon black, HAF (High Abrasion Furnace), SAF (Super Abrasion Furnace), ISAF (Intermediate SAF), ISAF-HM (Intermediate SAF-High Modulus), FEF (Fast Extrusion Furnace), MAF, GPF (General Purpose Furnace), and SRF (Semi-Reinforcing Furnace) are preferable.

For a rubber composition for a tire tread, carbon black having a CTAB surface area of 40-250 m²/g, a nitrogen adsorption specific surface area of 20-200 m²/g, and a particle size of 10-50 nm is preferably used, and carbon black having a CTAB surface area of 70-180 m²/g is further preferable. Examples thereof include N110, N220, N234, N299, N326, N330, N330T, N339, N343, N351 and the like according to ASTM standard. In addition, surface-treated carbon black obtained by attaching 0.1-50 wt % of silica to the surface of carbon black is also preferable.

Furthermore, it is also effective to combine several kinds of fillers such as combined use of carbon black and silica and the like. In a rubber composition for a tire tread, it is preferable to use carbon black alone or both carbon black and silica. In a rubber composition for a carcass or side wall, carbon black having a CTAB surface area of 20-60 m²/g, a particle size of 40-100 nm is preferably used. Examples thereof include N330, N339, N343, N351, N550, N568, N582, N630, N642, N660, N662, N754, N762 according to the ASTM standard.

While the amount of the filler to be used is not particularly limited, it is preferably 5-100 parts by weight per 100 parts by weight of a rubber component. When carbon black alone is used as a filler, the amount of the filler (=carbon black) to be used is preferably 30-80 parts by weight per 100 parts by weight of the rubber component. When silica and carbon black are used in combination as a filler in tread member use, the amount of the filler to be used is preferably 5-50 parts by weight per 100 parts by weight of a rubber component.

Examples of silica include silica with a CTAB specific surface area of 50-180 m²/g, silica with a nitrogen adsorption specific surface area of 50-300 m²/g. Examples of commercially available product of silica include “Nipsil (registered trade mark) AQ”, “Nipsil (registered trade mark) AQ-N” manufactured by Tosoh Silica Corporation, “Ultrasil (registered trade mark) VN3”, “Ultrasil (registered trade mark) VN3-G”, “Ultrasil (registered trade mark) 360”, “Ultrasil (registered trade mark) 7000” manufactured by Degussa, “Zeosil (registered trade mark) 115GR”, “Zeosil (registered trade mark) 1115 MP”, “Zeosil (registered trade mark) 1205 MP”, and “Zeosil (registered trade mark) Z85 MP” manufactured by Rhodia. In addition, (i) silica having pH 6-8, (ii) silica containing 0.2-1.5 wt % of sodium, (iii) spherical silica with a circularity of 1-1.3, (iv) silica surface-treated by silicone oil (e.g., dimethylsilicone oil), organic silicon compound containing ethoxysilyl group, alcohol (e.g., ethanol, polyethylene glycol) and the like, (v) a mixture of not less than two kinds of silica having different nitrogen adsorption specific surface areas may be used as a filler. For a rubber composition for a passenger car tread, silica is preferably used. The amount of silica in the rubber composition for a passenger car tread is preferably 10-120 parts by weight per 100 parts by weight of the rubber component. When silica is added, 5-50 parts by weight of carbon black is preferably added, and the weight ratio of silica/carbon black is preferably 0.7/1-1/0.1.

Examples of aluminum hydroxide include aluminum hydroxide having a nitrogen adsorption specific surface area of 5-250 m²/g and a DOP oil absorption of 50-100 ml/100 g.

An average particle size of ground bituminous coal is generally not more than 0.1 mm, preferably not more than 0.05 mm, more preferably not more than 0.01 mm. Even when ground bituminous coal having an average particle size exceeding 0.1 mm is used, hysteresis loss of the rubber composition cannot be lowered sufficiently and low fuel consumption may not be sufficiently improved. When the rubber composition of the present invention is used as a composition for an inner liner, even when ground bituminous coal having an average particle size exceeding 0.1 mm is used, the air-permeation resistance of the composition may not be improved sufficiently.

While the lower limit of the average particle size of the ground bituminous coal is not particularly limited, it is preferably not less than 0.001 mm. When it is less than 0.001 mm, the cost tends to be high. The average particle size of the ground bituminous coal is a mass standard average particle size calculated from the particle size distribution measured according to JIS Z 8815-1994.

The specific gravity of the ground bituminous coal is preferably not more than 1.6, more preferably not more than 1.5, further preferably not more than 1.3. When ground bituminous coal having a specific gravity exceeding 1.6 is used, the specific gravity of the whole rubber composition may increase, and improvement of low fuel consumption of tire may not be achieved sufficiently. The specific gravity of the ground bituminous coal is preferably not less than 0.5, more preferably not less than 1.0. When ground bituminous coal having a specific gravity of less than 0.5 is used, processability during kneading may be degraded.

When ground bituminous coal is used, the amount thereof is generally not less than 5 parts by weight, preferably not less than 10 parts by weight, generally not more than 70 parts by weight, preferably not more than 60 parts by weight, per 100 parts by weight of the rubber component. When the amount is less than 5 parts by weight, the effect of ground bituminous coal may not be obtained sufficiently, and when it exceeds 70 parts by weight, processability during kneading may be degraded.

### <Sulfur Component>

Examples of the sulfur component include powdered sulfur, precipitated sulfur, colloidal sulfur, insoluble sulfur, highly dispersible sulfur, morpholine disulfide, and tetramethylthiuram disulfide. Generally, powdered sulfur is preferable, and when the rubber composition of the present invention is used for the production of tire members having a large sulfur content such as belt member and the like, insoluble sulfur is preferable.

The amount of the sulfur component is preferably 0.01-30 parts by weight, more preferably 0.1-20 parts by weight, further preferably 0.1-10 parts by weight, per 100 parts by weight of the rubber component.

<Other Components>

The rubber composition may be produced by kneading other components in addition to the aforementioned compound (I), a rubber component, a filler and a sulfur component. Examples of other component include compound capable of bonding with silica, vulcanization accelerator, vulcanization supplement accelerator, resin, viscoelasticity improving agent, anti-aging agent, oil, wax, peptizing agent, retarder, compound having an oxyethylene unit, and catalyst (cobalt naphthenate etc.). Only one kind of any of other components may be used, or two or more kinds thereof may be used in combination.

When silica is used as a filler, compounds capable of bonding with silica such as silane coupling agent and the like are preferably used. Examples of the compound include bis(3-triethoxysilylpropyl)tetrasulfide (e.g., “Si-69” manufactured by Degussa), bis(3-triethoxysilylpropyl)disulfide (e.g., “Si-75” manufactured by Degussa), bis(3-diethoxymethylsilylpropyl)tetrasulfide, bis(3-diethoxymethylsilylpropyl)disulfide, 3-octanoylthiopropyltriethoxysilane (alias: “S-[3-(triethoxysilyl)propyl]octanethioate ester”, for example, “NXT silane” manufactured by General Electric Silicones), S-[3-{(2-methyl-1,3-propanedialkoxy)ethoxysilyl}propyl] octanethioate ester, S-[3-{(2-methyl-1,3-propanedialkoxy)methylsilyl}propyl]octanethioate ester, methyltrimethoxysilane, methyltriethoxysilane, methyltriacetoxysilane, methyltributoxysilane, ethyltrimethoxysilane, ethyltriethoxysilane, isobutyltrimethoxysilane, isobutyltriethoxysilane, n-octyltrimethoxysilane, n-octyltriethoxysilane, vinyltrimethoxysilane, vinyltriethoxysilane, vinyltris(methoxyethoxy)silane, phenyltrimethoxysilane, phenyltriethoxysilane, phenyltriacetoxysilane, 3-methacryloxypropyltrimethoxysilane, 3-methacryloxypropyltriethoxysilane, 3-aminopropyltrimethoxysilane, 3-aminopropyltriethoxysilane, N-(2-aminoethyl)-3-aminopropyltrimethoxysilane, N-(2-aminoethyl)-3-aminopropyltriethoxysilane, (3-glycidoxypropyl)trimethoxysilane, (3-glycidoxypropyl)triethoxysilane, 2-(3,4-epoxycyclohexyl)ethyltrimethoxysilane, 2-(3,4-epoxycyclohexyl)ethyltriethoxysilane, 3-isocyanatopropyltrimethoxysilane and 3-isocyanatopropyltriethoxysilane. Of these, bis(3-triethoxysilylpropyl)tetrasulfide (e.g., “Si-69” manufactured by Degussa), bis(3-triethoxysilylpropyl)disulfide (e.g., “Si-75” manufactured by Degussa), and 3-octanoylthiopropyltriethoxysilane (e.g., “NXT silane” manufactured by General Electric Silicones) are preferable.

While the timing of addition of a compound capable of bonding with silica is not particularly limited, it is preferably added to a rubber component simultaneously with silica. When silica and a compound capable of bonding with silica are used, the amount of the compound capable of bonding with silica is preferably 2-10 parts by weight, more preferably 7-9 parts by weight, per 100 parts by weight of silica. When a compound capable of bonding with silica is added, the addition temperature is preferably 80-200° C., more preferably 110-180° C.

When silica is used as a filler, it is preferable to use, in addition to a compound capable of bonding with silica, monovalent alcohols such as ethanol, butanol, octanol and the like; polyvalent alcohols such as ethylene glycol, diethylene glycol, triethylene glycol, polyethylene glycol, polypropylene glycol, pentaerythritol, polyether polyol and the like; N-alkylamine; amino acid; liquid polybutadiene having a carboxy-modified or amine-modified molecular terminal and the like.

Examples of the vulcanization accelerator include thiazole vulcanization accelerator, sulfenamide vulcanization accelerator, and guanidine vulcanization accelerator, which are described in GOMU KOGYO BINRAN (Rubber Industry Handbook) <fourth edition> (published by The Society of Rubber Science and Technology, Japan on Jan. 20, 1994) pp. 412-413.

Specific examples of the vulcanization accelerator include N-cyclohexyl-2-benzothiazolylsulfenamide (CBS), N-tert-butyl-2-benzothiazolylsulfenamide (BBS), N,N-dicyclohexyl-2-benzothiazolylsulfenamide (DCBS), 2-mercaptobenzothiazole (MBT), dibenzothiazyl disulfide (MBTS), and diphenylguanidine (DPG).

When carbon black alone is used as a filler, any of N-cyclohexyl-2-benzothiazolylsulfenamide (CBS), N-tert-butyl-2-benzothiazolylsulfenamide (BBS), N,N-dicyclohexyl-2-benzothiazolylsulfenamide (DCBS) and dibenzothiazyl disulfide (MBTS), and diphenylguanidine (DPG) are preferably used in combination as a vulcanization accelerator. When silica and carbon black are used in combination as a filler, any of N-cyclohexyl-2-benzothiazolylsulfenamide (CBS), N-tert-butyl-2-benzothiazolylsulfenamide (BBS), N,N-dicyclohexyl-2-benzothiazolylsulfenamide (DCBS) and dibenzothiazyl disulfide (MBTS), and diphenylguanidine (DPG) are preferably used in combination as a vulcanization accelerator.

While the ratio of sulfur component and vulcanization accelerator is not particularly limited, the weight ratio of sulfur component/vulcanization accelerator is preferably ⅕-5/1, more preferably ½-2/1. In addition, EV vulcanization setting the sulfur component/vulcanization accelerator ratio to not more than 1, which is a method of improving heat resistance, is preferably used in application particularly requiring improvement of heat resistance of a rubber member containing natural rubber as a main component.

Examples of the vulcanization supplement accelerator include zinc oxide, stearic acid, citraconimide compound, alkylphenol-sulfur chloride condensate, organic thiosulfate compound and a compound represented by the formula (III)

R¹⁶—S—S—R¹⁷—S—S—R¹⁸ (III)

wherein R¹⁷ is a C₂₋₁₀ alkanediyl group, and R¹⁶ and R¹⁸ are each independently a monovalent organic group containing a nitrogen atom. In the present invention, zinc oxide is encompassed in the concept of vulcanization supplement accelerator, and is not encompassed in the concept of the aforementioned filler.

As the vulcanization supplement accelerator, zinc oxide, stearic acid, and citraconimide compound are preferable, and zinc oxide and stearic acid are more preferable.

When zinc oxide is used, the amount thereof is preferably 0.01-20 parts by weight, more preferably 0.01-15 parts by weight, further preferably 0.01-10 parts by weight, per 100 parts by weight of the rubber component. When stearic acid is used, the amount thereof is preferably 0.01-20 parts by weight, more preferably 0.01-15 parts by weight, further preferably 0.01-10 parts by weight, per 100 parts by weight of the rubber component.

As the citraconimide compound, biscitraconimides is preferable since it is thermally stable, and superior in dispersibility in a rubber component. Specific examples thereof include 1,2-biscitraconimidomethylbenzene, 1,3-biscitraconimidomethylbenzene, 1,4-biscitraconimidomethylbenzene, 1,6-biscitraconimidomethylbenzene, 2,3-biscitraconimidomethyltoluene, 2,4-biscitraconimidomethyltoluene, 2,5-biscitraconimidomethyltoluene, 2,6-biscitraconimidomethyltoluene, 1,2-biscitraconimidoethylbenzene, 1,3-biscitraconimidoethylbenzene, 1,4-biscitraconimidoethylbenzene, 1,6-biscitraconimidoethylbenzene, 2,3-biscitraconimidoethyltoluene, 2,4-biscitraconimidoethyltoluene, 2,5-biscitraconimidoethyltoluene, 2,6-biscitraconimidoethyltoluene and the like.

Of the citraconimide compounds, 1,3-biscitraconimidomethylbenzene represented by the following formula is preferable, since it is particularly thermally stable, particularly superior in dispersibility in a rubber component, and affords a vulcanized rubber composition with high hardness (Hs) (reversion control).

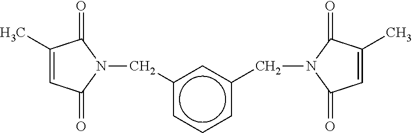

As a vulcanization supplement accelerator, an alkylphenol-sulfur chloride condensate represented by the formula (IV):

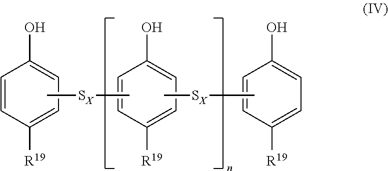

wherein n is an integer of 0-10, X is an integer of 2-4, and R¹⁹ is a C₅₋₁₂ alkyl group, is preferably used since a vulcanized rubber composition with high hardness (Hs) can be obtained.

Since alkylphenol-sulfur chloride condensate (IV) shows superior dispersibility in a rubber component, n is preferably an integer of 1-9.

When X exceeds 4, alkylphenol-sulfur chloride condensate (IV) tends to be thermally unstable, and when X is 1, alkylphenol-sulfur chloride condensate (IV) has low sulfur content (weight of sulfur). Since high hardness can be efficiently exhibited (reversion inhibition), X is preferably 2.

R¹⁹ is a C₅₋₁₂ alkyl group. Since alkylphenol-sulfur chloride condensate (IV) shows good dispersibility in a rubber component, R¹⁹ is preferably a C₆₋₉ alkyl group. Examples of the “C₅₋₁₂ alkyl group” and “C₆₋₉ alkyl group” include those in the aforementioned “C₁₋₁₂ alkyl group” which have a carbon number of 5-12 or a carbon number of 6-9.

Specific examples of the alkylphenol-sulfur chloride condensate (IV) include TACKIROL V200 wherein n is 0-10, X is 2, R¹⁹ is octyl, and sulfur content is 24 wt %, which is manufactured by TAOKA CHEMICAL COMPANY, LIMITED.

As a vulcanization supplement accelerator, a salt of an organic thiosulfate compound represented by the formula (V):

HO₃S—S—(CH₂)ₘ-S—SO₃H (V)

wherein m is an integer of 3-10
(hereinafter sometimes to be indicated as “organic thiosulfate compound salt (V)”) is preferably used since a vulcanized rubber composition with high hardness (Hs) can be obtained (reversion inhibition). Organic thiosulfate compound salt (V) containing crystal water may also be used. Examples of the organic thiosulfate compound salt (V) include lithium salt, potassium salt, sodium salt, magnesium salt, calcium salt, barium-salt, zinc salt, nickel salt, cobalt salt and the like, and potassium salt and sodium salt are preferable.

m is an integer of 3-10, preferably an integer of 3-6. When m is not more than 2, sufficient heat fatigue resistance tends to be unachieved, and when m is 11 or more, organic thiosulfate compound salt (V) may not show sufficient heat fatigue resistance improving effect.

As organic thiosulfate compound salt (V), a sodium salt 1 hydrate or a sodium salt 2 hydrate thereof is preferable since it is stable at ambient temperature and under normal pressure, organic thiosulfate compound salt (V) obtained from sodium thiosulfate is more preferable from the aspect of cost, and sodium 1,6-hexamethylenedithiosulfate 2 hydrate represented by the following formula is more preferable.

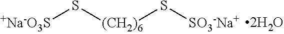

It is preferable to use a compound represented by the formula (III):

R¹⁶—S—S—R¹⁷—S—S—R¹⁸ (III)

wherein R¹⁷ is a C₂₋₁₀ alkanediyl group, and R¹⁶ and R¹⁸ are each independently a monovalent organic group containing a nitrogen atom,

as a vulcanization supplement accelerator, since it is dispersed well in a rubber component, and inserted in between —S_{X}— crosslinking of alkylphenol-sulfur chloride condensate (IV) when used in combination with alkylphenol-sulfur chloride condensate (IV) to form a hybrid crosslinking with alkylphenol-sulfur chloride condensate (IV).

R¹⁷ is a C₂₋₁₀ alkanediyl group, preferably a C₄₋₈ alkanediyl group, more preferably a linear C₄₋₈ alkanediyl group. R¹⁷ is preferably linear. When the carbon number of R¹⁷ is not more than 1, thermal stability may be low. When the carbon number of R¹⁷ is not less than 11, the distance between polymers via a vulcanization supplement accelerator becomes long, and the effect of addition of a vulcanization supplement accelerator may not be obtained. As “C₂₋₁₀ alkanediyl group” and “C₄₋₈ alkanediyl group”, the aforementioned “C₁₋₁₂ alkanediyl group” having a carbon number of 2-10, or a carbon number of 4-8 can be mentioned.

R¹⁶ and R¹⁸ are each independently a monovalent organic group containing a nitrogen atom. As the monovalent organic group containing a nitrogen atom, one containing at least one aromatic ring is preferable, and one containing an aromatic ring and a=N—C(═S)— group is more preferable. R¹⁶ and R¹⁸ may be the same or different, and preferably the same for the reasons of easy production and the like.

Examples of the compound (III) include 1,2-bis(dibenzylthiocarbamoyldithio)ethane, 1,3-bis(dibenzylthiocarbamoyldithio)propane, 1,4-bis(dibenzylthiocarbamoyldithio)butane, 1,5-bis(dibenzylthiocarbamoyldithio)pentane, 1,6-bis(dibenzylthiocarbamoyldithio)hexane, 1,7-bis(dibenzylthiocarbamoyldithio)heptane, 1,8-bis(dibenzylthiocarbamoyldithio)octane, 1,9-bis(dibenzylthiocarbamoyldithio)nonane, 1,10-bis(dibenzylthiocarbamoyldithio)decane and the like. Of these, 1,6-bis(dibenzylthiocarbamoyldithio)hexane is preferable since it is thermally stable, and superior in dispersibility in a rubber component.

Examples of the commercially available product of compound (III) include VULCUREN TRIAL PRODUCT KA9188, VULCUREN VP KA9188 (1,6-bis(dibenzylthiocarbamoyldithio)hexane) manufactured by Bayer, Ltd.

The rubber composition may contain organic compounds such as resorcinol and the like, resins such as resorcinol resin, modified resorcinol resin, cresol resin, modified cresol resin, phenol resin and modified phenol resin and the like. When it contains resorcinol or these resins, elongation at break and complex modulus of elasticity of the vulcanized rubber composition can be improved. When the rubber composition containing resorcinol or resins is used for the production of a rubber product to be in contact with a cord, the adhesiveness to the cord can be enhanced.

Examples of resorcinol include resorcinol manufactured by Sumitomo Chemical Company, Limited, and the like. Examples of the resorcinol resin include resorcinol-formaldehyde condensate. Examples of the modified resorcinol resin include a resorcinol resin having a partly alkylated repeat unit. To be specific, Penacolite resin B-18-S, B-20 manufactured by Indspec, SUMIKANOL 620 manufactured by TAOKA CHEMICAL COMPANY, LIMITED, R-6 manufactured by Uniroyal, SRF 1501 manufactured by Schenectady Chemical, Arofene 7209 manufactured by Ashland Inc. and the like can be mentioned.

Examples of the cresol resin include cresol-formaldehyde condensate. Examples of the modified cresol resin include a cresol resin wherein the terminal methyl group is modified into a hydroxy group, and a cresol resin having a partly alkylated repeat unit. To be specific, SUMIKANOL 610 manufactured by TAOKA CHEMICAL COMPANY, LIMITED, PR-X11061 manufactured by Sumitomo Bakelite Co., Ltd., and the like can be mentioned.

Examples of the phenol resin include phenol-formaldehyde condensate. Examples of the modified phenol resin include phenol resin modified using cashew oil, tall oil, flaxseed oil, various animal and vegetable oils, unsaturated fatty acid, rosin, alkylbenzene resin, aniline, melamine and the like.

Examples of other resin include methoxylated methylolmelamine resins such as “SUMIKANOL 507AP” manufactured by Sumitomo Chemical Company, Limited and the like; coumarone-inden resins such as coumarone resin NG4 (softening point 81-100° C.) manufactured by Nippon Steel chemical, “process resin AC5” (softening point 75° C.) manufactured by KOBE OIL CHEMICAL INDUSTRIAL Co., Ltd. and the like; terpene-based resins such as terpene resin, terpene-phenol resin, aromatic-modified terpene resin and the like; rosin derivatives such as “Nikanol (registered trade mark) A70” (softening point 70-90° C.) manufactured by MITSUBISHI GAS CHEMICAL COMPANY, INC. and the like; hydrogenated rosin derivative; novolac alkylphenol-based resin; resol alkylphenol-based resin; C5 petroleum resin; and liquid polybutadiene.

Examples of the viscoelasticity improving agent include N,N′-bis(2-methyl-2-nitropropyl)-1,6-hexanediamine (e.g., “Sumifine (registered trade mark) 1162” manufactured by Sumitomo Chemical Company, Limited), dithiouracil compound described in JP-A-63-23942, “TACKIROL (registered trade mark) AP”, “TACKIROL (registered trade mark) V-200” manufactured by TAOKA CHEMICAL COMPANY, LIMITED, alkylphenol-sulfur chloride condensate described in JP-A-2009-138148, bis(3-triethoxysilylpropyl)tetrasulfide (e.g., “Si-69” manufactured by Degussa), bis(3-triethoxysilylpropyl)disulfide (e.g., “Si-75” manufactured by Degussa), bis(3-diethoxymethylsilylpropyl)tetrasulfide, bis(3-diethoxymethylsilylpropyl)disulfide, octanethioate S-[3-(triethoxysilyl)propyl] ester, octanethioate S-[3-{(2-methyl-1,3-propanedialkoxy)ethoxysilyl}propyl] ester, octanethioate S-[3-{(2-methyl-1,3-propanedialkoxy)methylsilyl}propyl] ester, methyltrimethoxysilane, methyltriethoxysilane, methyltriacetoxysilane, methyltributoxysilane, ethyltrimethoxysilane, ethyltriethoxysilane, isobutyltrimethoxysilane, isobutyltriethoxysilane, n-octyltrimethoxysilane, n-octyltriethoxysilane, vinyltrimethoxysilane, vinyltriethoxysilane, vinyltri(methoxyethoxy)silane, phenyltrimethoxysilane, phenyltriethoxysilane, phenyltriacetoxysilane, 3-methacryloxypropyltrimethoxysilane, 3-methacryloxypropyltriethoxysilane, 3-aminopropyltrimethoxysilane, 3-aminopropyltriethoxysilane, N-(2-aminoethyl)-3-aminopropyltrimethoxysilane, N-(2-aminoethyl)-3-aminopropyltriethoxysilane, (3-glycidoxypropyl)trimethoxysilane, (3-glycidoxypropyl)triethoxysilane, 2-(3,4-epoxycyclohexyl)ethyltrimethoxysilane, 2-(3,4-epoxycyclohexyl)ethyltriethoxysilane, 3-isocyanatopropyltrimethoxysilane, 3-isocyanatopropyltriethoxysilane, 1,6-bis(dibenzylthiocarbamoyldithio)hexane (e.g., “KA9188” manufactured by Bayer, Ltd.), 1,6-hexamethylenedithiosulfate 2 sodium salt 2 hydrate, 1,3-bis(citraconimidomethyl)benzene (e.g., “Perkalink 900” manufactured by Flexsys), l-benzoyl-2-phenylhydrazide, 1-hydroxy-N′-(1-methylethylidene)-2-naphthoic acid hydrazide, 3-hydroxy-N′-(1-methylethylidene)-2-naphthoic acid hydrazide, carboxylic acid hydrazide derivatives such as l-hydroxy-N′-(l-methylpropylidene)-2-naphthoic acid hydrazide, 3-hydroxy-N′-(1-methylpropylidene)-2-naphthoic acid hydrazide, l-hydroxy-N′-(1,3-dimethylbutylidene)-2-naphthoic acid hydrazide, 3-hydroxy-N′-(1,3-dimethylbutylidene)-2-naphthoic acid hydrazide, 1-hydroxy-N′-(2-furylmethylene)-2-naphthoic acid hydrazide, 3-hydroxy-N′-(2-furylmethylene)-2-naphthoic acid hydrazide and the like described in JP-A-2004-91505, 3-hydroxy-N′-(1,3-dimethylbutylidene)-2-naphthoic acid hydrazide, 3-hydroxy-N′-(1,3-diphenylethylidene)-2-naphthoic acid hydrazide, 3-hydroxy-N′-(1-methylethylidene)-2-naphthoic acid hydrazide described in JP-A-2000-190704, bismercaptooxadiazole compound described in JP-A-2006-328310, pyrithione salt compound described in JP-A-2009-40898, and cobalt hydroxides described in JP-A-2006-249361.

Of these, N,N′-bis(2-methyl-2-nitropropyl)-1,6-hexanediamine (e.g., “Sumifine (registered trade mark) 1162” manufactured by Sumitomo Chemical Company, Limited), bis(3-triethoxysilylpropyl)tetrasulfide (e.g., “Si-69” manufactured by Degussa), bis(3-triethoxysilylpropyl)disulfide (e.g., “Si-75” manufactured by Degussa), 1,6-bis(dibenzylthiocarbamoyldithio)hexane (e.g., “KA9188” manufactured by Bayer, Ltd.), hexamethylenebisthiosulfate 2 sodium salt 2 hydrate, 1,3-bis(citraconimidomethyl)benzene (e.g., “Perkalink 900” manufactured by Flexsys), “TACKIROL (registered trade mark) AP”, and “TACKIROL (registered trade mark) V-200” manufactured by TAOKA CHEMICAL COMPANY, LIMITED are preferable.

Examples of the anti-aging agent include those described in “GOMU KOGYO BINRAN (Rubber Industry Handbook)<fourth edition>” pp. 436-443, edited by The Society of Rubber Science and Technology, Japan. As a preferable anti-aging agent, N-phenyl-N′-1,3-dimethylbutyl-p-phenylenediamine (abbreviation “6PPD”, for example, “Antigen (registered trade mark) 6C” manufactured by Sumitomo Chemical Company, Limited), reaction product of aniline and acetone (abbreviation “TMDQ”), poly(2,2,4-trimethyl-1,2-)dihydroquinoline) (e.g., “Antioxidant FR” manufactured by MATSUBARA SANGYO), synthesis wax (paraffin wax etc.), or plant-derived wax is preferably used.

When an anti-aging agent is used, the amount thereof is preferably 0.01-20 parts by weight, more preferably 0.01-15 parts by weight, further preferably 0.01-10 parts by weight, per 100 parts by weight of the rubber component.

Examples of the oil include process oil, vegetable fat and oil and the like. Examples of the process oil include paraffinic process oil, naphthenic process oil, and aromatic process oil. Examples of the commercially available product include aromatic oil (“NC-140” manufactured by Cosmo Oil Co., Ltd.), and process oil (“Diana process PS32” manufactured by Idemitsu Kosan Co., Ltd.).

Examples of the wax include “SANNOC (registered trade mark) wax” manufactured by OUCHI SHINKO CHEMICAL INDUSTRIAL CO., LTD., “OZOACE-0355” manufactured by Nippon Seiro Co., Ltd., and the like.

A peptizing agent is not particularly limited as long as it is generally used in the field of rubber. Examples thereof include aromatic mercaptan-based peptizing agent, aromatic disulfide-based peptizing agent, aromatic mercaptan metal salt-based peptizing agent, described in “GOMU KOGYO BINRAN (Rubber Industry Handbook)<fourth edition>” pp. 446-449 edited by The Society of Rubber Science and Technology, Japan. Of these, dixylyl disulfide and o,o′-dibenzamidodiphenyl disulfide (“NOCTIZER SS” manufactured by OUCHI SHINKO CHEMICAL INDUSTRIAL CO., LTD.) are preferable. Only one kind of peptizing agent may be used, or two or more kinds thereof may be used in combination.

While the amount of the peptizing agent to be used is not particularly limited, it is preferably 0.01-1 part by weight, more preferably 0.05-0.5 parts by weight, per 100 parts by weight of the rubber component.

Examples of the retarder include phthalic anhydride, benzoic acid, salicylic acid, N-nitrosodiphenylamine, N-(cyclohexylthio)phthalimide (CTP), sulfonamide derivative, diphenylurea, bis(tridecyl)pentaerythritol diphosphite and the like, and N-(cyclohexylthio)phthalimide (CTP) is preferably used.

While the amount of the retarder to be used is not particularly limited, it is preferably 0.01-1 part by weight, more preferably 0.05-0.5 parts by weight, per 100 parts by weight of the rubber component.

The rubber composition of the present invention may contain a compound having an oxyethylene unit having a structure represented by the formula: —O—(CH₂—CH₂—O)_{q}—H wherein q is an integer of 1 or more. In the above-mentioned formula, q is preferably 2 or more, more preferably 3 or more. In addition, q is preferably 16 or less, more preferably 14 or less. When q is 17 or more, compatibility with a rubber component and reinforcing performance tend to decrease.

The position of the oxyethylene unit in a compound having an oxyethylene unit may be main chain, or terminal, or side chain. From the aspects of the sustainability of the effect of preventing static electricity accumulation on the surface of the obtained tire and reduction of electrical resistance, of the compounds having an oxyethylene unit, a compound having an oxyethylene unit at least on the side chain is preferable.

Examples of a compound having an oxyethylene unit in the main chain include polyoxyethylene alkyl ether, polyoxyethylene alkyl phenyl ether, monoethylene glycol, diethylene glycol, triethylene glycol, polyoxyethylene sorbitan fatty acid ester, polyoxyethylene polyoxypropylene alkyl ether, polyoxyethylene alkylamine, polyoxyethylene styrenated alkyl ether, polyoxyethylene alkylamide and the like.

When a compound having an oxyethylene unit at least in the side chain is used, the number of oxyethylene unit is preferably not less than 4, more preferably not less than 8, per 100 carbon atoms constituting the main chain. When the number of oxyethylene unit is not more than 3, the electrical resistance tends to increase. The number of oxyethylene unit is preferably not more than 12, more preferably not more than 10. When the number of oxyethylene unit is not less than 13, compatibility with a rubber component and reinforcing performance tend to decrease.

When a compound having an oxyethylene unit at least in the side chain is used, the main chain thereof is preferably mainly constituted of polyethylene, polypropylene or polystyrene.

### <Rubber Composition>

When a rubber composition is used for, for example, tires, the rubber composition is required to have various properties to achieve low fuel consumption, high-speed resistance, good dry-wet grip performance and the like of the tire. A rubber component and the like are appropriately selected according to the required property, whereby a rubber composition suitable for each use can be prepared.

A rubber component in a rubber composition preferable for a tread member suitable for truck, bus, light truck, and large tire for construction is preferably natural rubber alone or a blend of natural rubber as a main component, and SBR and/or BR as sub component(s). The filler is preferably carbon black alone, or a blend of silica as a main component, and carbon black as a sub component. Furthermore, viscoelasticity improving agents such as N,N′-bis(2-methyl-2-nitropropyl)-1,6-hexanediamine (e.g., “Sumifine (registered trade mark) 1162” manufactured by Sumitomo Chemical Company, Limited), bis(3-triethoxysilylpropyl)tetrasulfide (e.g., “Si-69” manufactured by Degussa), bis(3-triethoxysilylpropyl)disulfide (e.g., “Si-75” manufactured by Degussa), 1,6-bis(dibenzylthiocarbamoyldithio)hexane (e.g., “KA9188” manufactured by Bayer, Ltd.), hexamethylenebisthiosulfate 2 sodium salt 2 hydrate, 1,3-bis(citraconimidomethyl)benzene (e.g., “Perkalink 900” manufactured by Flexsys), “TACKIROL (registered trade mark) AP”, “TACKIROL (registered trade mark) V-200” manufactured by TAOKA CHEMICAL COMPANY, LIMITED and the like are preferably used.

A rubber component in a rubber composition preferable for a tread member suitable for passenger car tires is preferably solution-polymerized SBR alone having a molecular terminal modified with a silicon compound, or a blend of the aforementioned terminal modified solution-polymerized SBR as a main component and, at least one kind selected from the group consisting of unmodified solution-polymerized SBR, emulsion-polymerized SBR, natural rubber and BR as a sub component. The filler is preferably a blend of silica as a main component, and carbon black as a sub component. Furthermore, viscoelasticity improving agents such as N,N′-bis(2-methyl-2-nitropropyl)-1,6-hexanediamine (e.g., “Sumifine (registered trade mark) 1162” manufactured by Sumitomo Chemical Company, Limited), bis(3-triethoxysilylpropyl)tetrasulfide (e.g., “Si-69” manufactured by Degussa), bis(3-triethoxysilylpropyl)disulfide (e.g., “Si-75” manufactured by Degussa), 1,6-bis(dibenzylthiocarbamoyldithio)hexane (e.g., “KA9188” manufactured by Bayer, Ltd.), hexamethylenebisthiosulfate 2 sodium salt 2 hydrate, 1,3-bis(citraconimidomethyl)benzene (e.g., “Perkalink 900” manufactured by Flexsys), “TACKIROL (registered trade mark) AP”, “TACKIROL (registered trade mark) V-200” manufactured by TAOKA CHEMICAL COMPANY, LIMITED and the like are preferably used.

A rubber component in a rubber composition preferable for a side wall member is preferably a blend of BR as a main component and, at least one kind selected from the group consisting of unmodified solution-polymerized SBR, emulsion-polymerized SBR and natural rubber as a sub component. The filler is preferably carbon black alone or a blend of carbon black as a main component, and silica as a sub component. Furthermore, viscoelasticity improving agents such as N,N′-bis(2-methyl-2-nitropropyl)-1,6-hexanediamine (e.g., “Sumifine (registered trade mark) 1162” manufactured by Sumitomo Chemical Company, Limited), bis(3-triethoxysilylpropyl)tetrasulfide (e.g., “Si-69” manufactured by Degussa), bis(3-triethoxysilylpropyl)disulfide (e.g., “Si-75” manufactured by Degussa), 1,6-bis(dibenzylthiocarbamoyldithio)hexane (e.g., “KA9188” manufactured by Bayer, Ltd.), hexamethylenebisthiosulfate 2 sodium salt 2 hydrate, 1,3-bis(citraconimidomethyl)benzene (e.g., “Perkalink 900” manufactured by Flexsys), “TACKIROL (registered trade mark) AP”, “TACKIROL (registered trade mark) V-200” manufactured by TAOKA CHEMICAL COMPANY, LIMITED and the like are preferably used.

A rubber component in a rubber composition preferable for a carcass or belt member is preferably natural rubber alone, or a blend of natural rubber as a main component and BR as a sub component. The filler is preferably carbon black alone or a blend of carbon black as a main component, and silica as a sub component. Furthermore, viscoelasticity improving agents such as N,N′-bis(2-methyl-2-nitropropyl)-1,6-hexanediamine (e.g., “Sumifine (registered trade mark) 1162” manufactured by Sumitomo Chemical Company, Limited), bis(3-triethoxysilylpropyl)tetrasulfide (e.g., “Si-69” manufactured by Degussa), bis(3-triethoxysilylpropyl)disulfide (e.g., “Si-75” manufactured by Degussa), 1,6-bis(dibenzylthiocarbamoyldithio)hexane (e.g., “KA9188” manufactured by Bayer, Ltd.), hexamethylenebisthiosulfate 2 sodium salt 2 hydrate, 1,3-bis(citraconimidomethyl)benzene (e.g., “Perkalink 900” manufactured by Flexsys), “TACKIROL (registered trade mark) AP”, “TACKIROL (registered trade mark) V-200” manufactured by TAOKA CHEMICAL COMPANY, LIMITED and the like are preferably used.

### <Production of Rubber Composition>

The rubber composition of the present invention can be produced by kneading compound (I), a rubber component and a filler, and other components as necessary.

The rubber composition of the present invention obtained by kneading the aforementioned components and, further, a sulfur component (hereinafter sometimes to be referred to as “rubber composition containing sulfur component”) is preferably produced via a step of kneading a rubber component and a filler (hereinafter sometimes to be referred to as “step 1”), and then a step of kneading the rubber composition obtained in step 1 and a sulfur component (hereinafter sometimes to be referred to as “step 2”). Furthermore, a pre-kneading step for masticating the rubber component may be included before step 1 (i.e., kneading rubber component and filler) to facilitate processing of the rubber component.

In the production of a rubber composition containing a sulfur component, the total amount of compound (I) may be kneaded with a rubber component and the like in any of the pre-kneading step, step 1 and step 2, or compound (I) may be divided and kneaded with a rubber component and the like in at least two steps of pre-kneading step-step 2. Compound (I) is preferably kneaded with a rubber component and the like before step 2. When a pre-kneading step is performed, it is preferable to knead the total amount of compound (I) with a rubber component in the pre-kneading step, or divide compound (I) and knead with a rubber component in both the pre-kneading step and step 1.

Compound (I) may be supported on a carrier in advance, and then kneaded with a rubber component and the like. Examples of the carrier include the fillers exemplified earlier, and the inorganic fillers and reinforcing agents described in “GOMU KOGYO BINRAN (Rubber Industry Handbook)<fourth edition>”, pp. 510-513, edited by The Society of Rubber Science and Technology, Japan. As the carrier, carbon black, silica, calcined clay, and aluminum hydroxide are preferable. The amount of the carrier to be used is not particularly limited, and is preferably 10-1,000 parts by weight, more preferably 100-1,000 parts by weight, further preferably 200-1,000 parts by weight, per 100 parts by weight of compound (I).

When zinc oxide is added, it is preferably kneaded with a rubber component and the like in step 1. When a vulcanization accelerator is added, it is preferably kneaded with a rubber component and the like in step 2. When a peptizing agent is added, it is preferably kneaded with a rubber component and the like in step 1. When a pre-kneading step is performed, it is preferable to knead the total amount of the peptizing agent with a rubber component in the pre-kneading step or divide the peptizing agent and knead with the rubber component in both the pre-kneading step and step 1. When a retarder is added, it is preferably kneaded with a rubber component and the like in step 2.

For kneading in step 1, for example, internal mixer including Banbury mixer, open kneader, pressure kneader, extruder, injection molder and the like can be used. The discharging temperature of the rubber composition after kneading in step 1 is preferably not more than 200° C., more preferably 120-180° C.

For kneading in step 2, for example, open roll, calendar and the like can be used. The kneading temperature (temperature rubber composition being kneaded) in step 2 is preferably 60-120° C.

### <Vulcanized Rubber Composition>

A vulcanized rubber composition can be produced by vulcanizing a rubber composition containing a sulfur component. A vulcanized rubber composition may also be produced by vulcanizing the aforementioned rubber composition after processing into a particular shape.

The vulcanizing temperature is preferably 120-180° C. Those of ordinary skill in the art can appropriately determine the vulcanizing time according to the composition of the rubber composition. Vulcanization is generally performed under normal pressure or under pressure.

### <Application>

The rubber composition and vulcanized rubber composition of the present invention are useful for producing various products. As the products obtained from the rubber composition and vulcanized rubber composition of the present invention, a vulcanized tire and a tire member are preferable. Examples of the tire member include a tire belt member containing a vulcanized rubber composition of the present invention and a steel cord, a tire carcass member containing a vulcanized rubber composition of the present invention and a carcass fiber cord, a tire side a wall member, a tire inner liner member, a tire cap tread member and a tire under tread member.

A vulcanized tire is produced by first producing tire members, combining these to produce a green tire, and vulcanizing the green tire. A tire produced using the rubber composition of the present invention has a low loss factor (tan δ) and can achieve low fuel consumption.

The vulcanized rubber composition of the present invention can be used not only for the above-mentioned tire application but also various vibration-proof rubbers. Examples of the vibration-proof rubber include automotive vibration-proof rubbers such as engine mount, strut mount, bush, exhaust hanger and the like. The vibration-proof rubber can be produced by first processing a rubber composition containing a sulfur component into a given shape and then vulcanizing same.

### EXAMPLES

While the present invention is specifically explained in the following by referring to Examples, Experimental Example, Production Example and the like, the present invention is not limited to them.

### Production Example 1: Production of Compound (I-1)

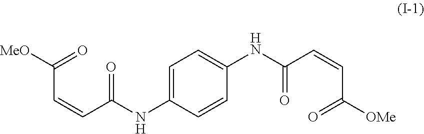 [SMILES: COC(=O)/C=C\C(=O)Nc1ccc(NC(=O)/C=C\C(=O)OC)cc1]

Under a nitrogen atmosphere, 1,4-phenylenediamine (100.4 g, 0.928 mol) and methanol (1000 ml) were charged in a reactor and, after dissolution, the methanol solution was cooled to 0-10° C. Under ice-cooling, maleic anhydride (227.2 g, 2.320 mol) was added thereto by small portions over about 1 hr while maintaining the temperature at 0-10° C. The mixture was gradually warmed to room temperature, methanol (200 ml) was added for further stirring, and the mixture was stirred in situ for 2 hr. After completion of the reaction, precipitated crystals were collected by filtration, and washed twice with methanol (100 ml) to give an intermediate (403 g).

Under a nitrogen atmosphere, methanol (1000 ml) was charged in another reactor, and methanol in a flask was cooled to −10° C. While maintaining the solution temperature at 0° C. or below, thionyl chloride (242.9 g, 2.042 mol) was added dropwise to the flask. To the methanol mixture was added the earlier intermediate (403 g) as a solid over 15 min and, after completion of addition, the temperature of the methanol mixture was raised to room temperature with stirring. Thereafter, the mixture was heated to 30° C., and the slurry solution was stirred for 5 hr. After stirring, the slurry solution was cooled to 20° C. and the reaction product was collected by filtration. The filtered residue was washed 3 times with methanol (150 ml) and dried to give the object compound (I-1) (260.6 g, 0.785 mol, yield 84.6%) as a pale-yellow powder.

¹H-NMR (6, ppm, DMSO-d₆): 3.67 (6H, s, 0-Me), 6.40 (2H, d, —CH═CH—COOH, j=11.7 Hz), 6.49 (2H, d, —CH═CH—CONH, j=11.7 Hz), 7.56 (4H, s, Ar—H), 10.26 (2H, s, —NHCO).

FD-MS (+):332 [M]+

### Production Example 2: Production of Compound (a)

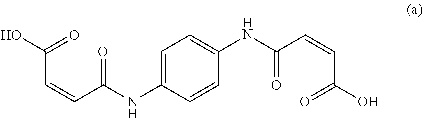 [SMILES: O=C(O)/C=C\C(=O)Nc1ccc(NC(=O)/C=C\C(=O)O)cc1]

Under a nitrogen atmosphere, 1,4-phenylenediamine (200.0 g, 1.85 mol) and methanol (4000 ml) were charged in a 5 L reactor, and the mixture was cooled with water. At room temperature, maleic anhydride (453 g, 4.62 mol) was added by small portions over 75 min, and the mixture was stirred at room temperature overnight. After completion of the reaction, a yellow-orange precipitate was collected by filtration and washed 3 times with methanol (600 ml) to give the object compound (a) (498.7 g, 1.64 mol, yield 88.7%).

¹H-NMR (δ, ppm, DMSO-d₆): 6.38 (4H, dd, —CH═CH—), 7.59 (4H, s, Ar—H), 10.44 (2H, s, Ar—NH), 13.18 (2H, s, —COOH)

### Production Example 3: Production of Compound (b)

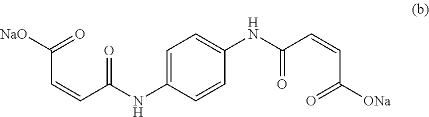

Under a nitrogen atmosphere, compound (a) (498.7 g, 1.64 mol) and water (2200 ml) were charged in a 5 L reactor, and the mixture was cooled with ice water. Under cooling with ice water, a solution prepared from sodium hydroxide (148 g, 3.70 mol) and water (600 ml) was added dropwise to a slurry solution of compound (a) over 1 hr. After dropwise addition, the reaction mixture was allowed to warm to room temperature, and stirred for 1 hr at around 20° C. After completion of the reaction, 2-propanol (2000 ml) was added dropwise to the reaction mixture while cooling with ice water, and the mixture was stirred at 10° C. or below for 1 hr. The precipitate was collected by filtration, washed with 2-propanol (400 ml) and dried under reduced pressure to give the object compound (b) (571.8 g, 1.64 mol, yield 88.7%) as a yellow powder.

¹H-NMR (δ, ppm, D₂O): 6.09 (2H, d, —CH═CH—CO₂Na), 6.46 (2H, d, —CH═CH—CONH), 7.50 (4H, s, Ar—H)

### Example 1: Production of Vulcanized Rubber Composition

### <Step 1>

Using Banbury mixer (600 ml Labo Plastomill manufactured by Toyo Seiki Ltd.), natural rubber (RSS #1) (100 parts by weight), HAF (trade name “Asahi #70” manufactured by Asahi Carbon Co., Ltd.) (45 parts by weight), stearic acid (3 parts by weight), zinc oxide (5 parts by weight), an anti-aging agent (N-phenyl-N′-1,3-dimethylbutyl-p-phenylenediamine, trade name “Antigen (registered trade mark) 6C” manufactured by Sumitomo Chemical Company, Limited) (1 part by weight) and compound (I-1) (1 part by weight) obtained in the above-mentioned Production Example 1 were kneaded to give a rubber composition. This step was performed by kneading at a mixer rotation speed of 50 rpm for 5 min after adding various components, with apparatus temperature at the start of kneading of 120° C., and the temperature of the rubber composition at discharge was 160-170° C.

### <Step 2>

The rubber composition obtained in step 1, a vulcanization accelerator (N-cyclohexyl-2-benzothiazolylsulfenamide) (1 part by weight) and powdered sulfur (2 parts by weight) were kneaded in an open roll at 60-80° C. to give a rubber composition.

### <Vulcanization>

The rubber composition obtained in step 2 was heated at 145° C. to give a vulcanized rubber composition. Vulcanization was performed for a time period of the 90% vulcanizing time (tc(90)) obtained by rheometer measurement according to JIS K 6300-2 plus 5 min.

### Example 2: Production of Vulcanized Rubber Composition

In the same manner as in Example 1 except that the amount of compound (I-1) to be added was changed from 1 part by weight to 0.5 parts by weight, a vulcanized rubber composition was obtained.

### Example 3: Production of Vulcanized Rubber Composition

In the same manner as in Example 1 except that the amount of compound (I-1) to be added was changed from 1 part by weight to 2 parts by weight, a vulcanized rubber composition was obtained.

### Example 4: Production of Vulcanized Rubber Composition

In the same manner as in Example 1 except that the amount of compound (I-1) to be added was changed from 1 part by weight to 4 parts by weight, a vulcanized rubber composition was obtained.

### Comparative Example 1: Production of Vulcanized Rubber Composition

In the same manner as in Example 1 except that compound (a) obtained in the same manner as in the above-mentioned Production Example 2 was used instead of compound (I-1), a vulcanized rubber composition was obtained.

### Comparative Example 2: Production of Vulcanized Rubber Composition

In the same manner as in Example 1 except that compound (b) obtained in the above-mentioned Production Example 3 was used instead of compound (I-1), a vulcanized rubber composition was obtained.

### Reference Example 1: Production of Vulcanized Rubber Composition

In the same manner as in Example 1 except that compound (I-1) was not used, a vulcanized rubber composition was obtained.

The formulations of Examples 1-4, Comparative Examples 1 and 2 and Reference Example 1 are shown in Table 1.

TABLE 1

| | | Ex. 1 | Ex. 2 | Ex. 3 | Ex. 4 | Com. Ex. 1 | Com. Ex. 2 | Ref. Ex. 1 |
|---|---|---|---|---|---|---|---|---|
| | | parts by | parts by | parts by | parts by | parts by | parts by | parts by |
| | component | weight | weight | weight | weight | weight | weight | weight |
| step | natural rubber | 100 | 100 | 100 | 100 | 100 | 100 | 100 |
| 1 | carbon black | 45 | 45 | 45 | 45 | 45 | 45 | 45 |
| | zinc oxide | 5 | 5 | 5 | 5 | 5 | 5 | 5 |
| | stearic acid | 3 | 3 | 3 | 3 | 3 | 3 | 3 |
| | anti-aging agent | 1 | 1 | 1 | 1 | 1 | 1 | 1 |
| | compound (I-1) | 1 | 0.5 | 2 | 4 | 0 | 0 | 0 |
| | compound (a) | 0 | 0 | 0 | 0 | 1 | 0 | 0 |
| | compound (b) | 0 | 0 | 0 | 0 | 0 | 1 | 0 |
| step | sulfur | 2 | 2 | 2 | 2 | 2 | 2 | 2 |
| 2 | vulcanization | 1 | 1 | 1 | 1 | 1 | 1 | 1 |
| | accelerator | | | | | | | |

### Experimental Example 1: Measurement of Loss Factor Lowering Effect

The viscoelasticity property of the vulcanized rubber composition obtained in Examples 1-4, Comparative Examples 1 and 2, and Reference Example 1 was measured under the following conditions by using a viscoelasticity analyzer manufactured by Ueshima Seisakusho Co., Ltd., and the loss factor (tan δ) thereof at 60° C. was determined. In the following, the “loss factor (tan δ) of the vulcanized rubber composition obtained in Reference Example 1 at 60° C.” and the like are abbreviated as “loss factor of Reference Example 1” and the like.

measurement temperature: −5° C.-80° C.

temperature rise rate: 2° C./min

initial strain: 10%

dynamic strain: 2.5%

frequency: 10 Hz

Using the determined loss factor of Reference Example 1 and the like and by the following formula (1), the loss factor lowering effect (%) of the vulcanized rubber compositions obtained in Examples and Comparative Examples was calculated. The results are shown in Table 2.

loss factor lowering effect (%)=100×(loss factor of Reference Example 1−loss factor of Example or Comparative Example)/(loss factor of Reference Example 1) (1)

TABLE 2

| | | | | | Com. | Com. | Ref. |
|---|---|---|---|---|---|---|---|
| | Ex. 1 | Ex. 2 | Ex. 3 | Ex. 4 | Ex. 1 | Ex. 2 | Ex. 1 |
| loss factor lowering | 16 | 10 | 21 | 24 | −4 | −5 | 0 |
| effect (%) | | | | | | | |

When compound (I-1) (ester) was used, the loss factor lowering effect became plus. However, when compound (a) (carboxylic acid) and compound (b) (carboxylic acid salt) similar thereto were used, the loss factor lowering effect became minus. That is, it was clarified that the loss factor of vulcanized rubber compositions can be lowered by using compound (I-1), but the loss factor of vulcanized rubber composition was conversely increased by using compound (a) or compound (b) similar thereto.

### Example 5: Production of Vulcanized Tire

By covering a steel cord, which is subjected to a brass plating treatment, with the rubber composition obtained in Example 1, a belt is obtained. Using the obtained belt, a green tire is formed according to a general production method, and the obtained green tire is heated and pressurized in a vulcanizer to give a vulcanized tire.

### Example 6: Production of Vulcanized Tire

The rubber composition obtained in Example 1 is extrusion processed to give a tread member. Using the obtained tread member, a green tire is formed according to a general production method, and the obtained green tire is heated and pressurized in a vulcanizer to give a vulcanized tire.

### Example 7: Production of Vulcanized Tire

The rubber composition obtained in Example 1 is extrusion processed to prepare a rubber composition having a shape corresponding to the carcass shape, which is adhered to the top and bottom of a polyester carcass fiber cord to a give carcass. Using the obtained carcass, a green tire is formed according to a general production method, and the obtained green tire is heated and pressurized in a vulcanizer to give a vulcanized tire.

### Example 8: Production of Rubber Composition

In the same manner as in Example 1 except that N-(cyclohexylthio)-phthalimide (CTP) (0.2 parts by weight) is further added, a rubber composition is obtained.

### Example 9: Production of Rubber Composition

In the same manner as in Example 1 except that o,o′-dibenzamidodiphenyl disulfide (“NOCTIZER SS” manufactured by OUCHI SHINKO CHEMICAL INDUSTRIAL CO., LTD.) (0.2 parts by weight) is further added in step 1, a rubber composition is obtained.

### Example 10: Production of Vulcanized Rubber Composition

### <Step 1>

Using Banbury mixer (600 ml Labo Plastomill manufactured by Toyo Seiki Ltd.), styrene-butadiene copolymer rubber SBR #1502 (manufactured by Sumitomo Chemical Company, Limited) (100 parts by weight), ISAF-HM (trade name “Asahi #80” manufactured by Asahi Carbon Co., Ltd.) (45 parts by weight), stearic acid (2 parts by weight), zinc oxide (3 parts by weight), compound (I-1) (1 part by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (1 part by weight) and wax (“OZOACE-0355” manufactured by Nippon Seiro Co., Ltd.) (2 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N-cyclohexyl-2-benzothiazolylsulfenamide (CBS)) (3 parts by weight) and powdered sulfur (2 parts by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for a cap tread.

### Example 11: Production of Rubber Composition

### <Step 1>

Using Banbury mixer (600 ml Labo Plastomill manufactured by Toyo Seiki Ltd.), styrene-butadiene copolymer rubber SBR #1502 (manufactured by Sumitomo Chemical Company, Limited) (100 parts by weight), ISAF-HM (trade name “Asahi #80” manufactured by Asahi Carbon Co., Ltd.) (35 parts by weight), stearic acid (2 parts by weight), zinc oxide (3 parts by weight), compound (I-1) (1 part by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (1 part by weight) and wax (“OZOACE-0355” manufactured by Nippon Seiro Co., Ltd.) (2 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N-cyclohexyl-2-benzothiazolylsulfenamide (CBS)) (2 parts by weight), a vulcanization accelerator (diphenylguanidine (DPG)) (0.5 parts by weight), a vulcanization accelerator (dibenzothiazyl disulfide (MBTS)) (0.8 parts by weight) and powdered sulfur (1 parts by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for an under tread.

### Example 12: Production of Vulcanized Rubber Composition

### <Step 1>

Using Banbury mixer (600 ml Labo Plastomill manufactured by Toyo Seiki Ltd.), natural rubber (RSS #1) (100 parts by weight), HAF (trade name “Asahi #70” manufactured by Asahi Carbon Co., Ltd.) (45 parts by weight), stearic acid (3 parts by weight), zinc oxide (5 parts by weight), compound (I-1) (1 part by weight), hydrated silica (“Nipsil (registered trade mark) AQ” manufactured by Tosoh Silica Corporation) (10 parts by weight), an anti-aging agent (“Antioxidant FR” manufactured by MATSUBARA SANGYO) (2 parts by weight), resorcinol (2 parts by weight) and cobalt naphthenate (2 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N,N-dicyclohexyl-2-benzothiazolylsulfenamide (DCBS)) (1 part by weight), insoluble sulfur (6 parts by weight) and methoxylated methylolmelamine resin (“SUMIKANOL 507AP” manufactured by Sumitomo Chemical Company, Limited) (3 parts by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for a belt.

### Example 13: Production of Vulcanized Rubber Composition

### <Step 1>

Using Banbury mixer (600 ml Labo Plastomill manufactured by Toyo Seiki Ltd.), halogenated butyl rubber (“Br-IIR 2255” manufactured by Exxon Mobil Corporation) (100 parts by weight), GPF (60 parts by weight), stearic acid (1 part by weight), zinc oxide (3 parts by weight), compound (I-1) (1 part by weight) and process oil (“Diana process PS32” manufactured by Idemitsu Kosan Co., Ltd.) (10 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, an anti-aging agent (reaction product of aniline and acetone (TMDQ)) (1 part by weight), a vulcanization accelerator (dibenzothiazyl disulfide (MBTS)) (1 part by weight) and powdered sulfur (2 parts by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for an inner liner.

### Example 14: Production of Vulcanized Rubber Composition

### <Step 1>

Using Banbury mixer (600 ml Labo Plastomill manufactured by Toyo Seiki Ltd.), natural rubber (RSS #3) (40 parts by weight), polybutadiene rubber (“BR150B” manufactured by Ube Industries, Ltd.) (60 parts), FEF (50 parts by weight), stearic acid (2.5 parts by weight), zinc oxide (3 parts by weight), compound (I-1) (1 part by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (2 parts by weight), aromatic oil (“NC-140” manufactured by Cosmo Oil Co., Ltd.) (10 parts by weight) and wax (“SANNOC (registered trade mark) wax” manufactured by OUCHI SHINKO CHEMICAL INDUSTRIAL CO., LTD.) (2 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N-tert-butyl-2-benzothiazolylsulfenamide (BBS)) (0.75 parts by weight) and powdered sulfur (1.5 parts by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for a side wall.

### Example 15: Production of Vulcanized Rubber Composition

### <Step 1>

Using Banbury mixer (600 ml Labo Plastomill manufactured by Toyo Seiki Ltd.), natural rubber (TSR20) (70 parts by weight), styrene-butadiene copolymer rubber SBR #1502 (manufactured by Sumitomo Chemical Company, Limited) (30 parts by weight), N339 (manufactured by Mitsubishi Chemical Corporation) (60 parts by weight), stearic acid (2 parts by weight), zinc oxide (5 parts by weight), process oil (“Diana process PS32” manufactured by Idemitsu Kosan Co., Ltd.) (7 parts by weight) and compound (I-1) (1 part by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N-tert-butyl-2-benzothiazolylsulfenamide (BBS)) (1 part by weight), powdered sulfur (3 parts by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (1 part by weight) and an anti-aging agent (reaction product of aniline and acetone (TMDQ)) (1 part by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for a carcass.

### Example 16: Production of Vulcanized Rubber Composition

### <Step 1>

Using Banbury mixer (600 ml Labo Plastomill manufactured by Toyo Seiki Ltd.), styrene-butadiene copolymer rubber SBR #1500 (manufactured by JSR) (100 parts by weight), silica (trade name: “Ultrasil (registered trade mark) VN3-G” manufactured by Degussa) (78.4 parts by weight), carbon black (trade name “N-339” manufactured by Mitsubishi Chemical Corporation) (6.4 parts by weight), a silane coupling agent (bis(3-triethoxysilylpropyl)tetrasulfide, trade name “Si-69” manufactured by Degussa) (6.4 parts by weight), process oil (trade name “NC-140” manufactured by Cosmo Oil Co., Ltd.) (47.6 parts by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (1.5 parts by weight), zinc oxide (2 parts by weight), stearic acid (2 parts by weight), and compound (I-1) (3 parts by weight) are kneaded to give a rubber composition. This step is performed within a temperature range of 70-120° C. by kneading at a mixer rotation speed of 80 rpm for 5 min after adding respective components, and successively kneading at a mixer rotation speed of 100 rpm for 5 min.

### <Step 2>

The rubber composition obtained in step 1, a vulcanization accelerator (N-cyclohexyl-2-benzothiazolylsulfenamide (CBS)) (1 part by weight), a vulcanization accelerator (diphenylguanidine (DPG)) (1 part by weight), wax (trade name “SANNOC (registered trade mark) N”, manufactured by OUCHI SHINKO CHEMICAL INDUSTRIAL CO., LTD.) (1.5 parts by weight) and powdered sulfur (1.4 parts by weight) are kneaded in an open roll at 30-80° C. to give a rubber composition.

### <Vulcanization>

The obtained rubber composition is heated at 160° C. to give a vulcanized rubber composition. Such vulcanized rubber composition is preferable for a cap tread.

### Example 17: Production of Vulcanized Rubber Composition

In the same manner as in Example 16 except that solution-polymerized SBR (“Asaprene (registered trade mark)” manufactured by Asahi Kasei Chemicals Corporation) is used instead of styrene-butadiene copolymer rubber SBR #1500 (manufactured by JSR), a rubber composition is obtained. The obtained rubber composition is heated at 160° C. to give a vulcanized rubber composition. Such vulcanized rubber composition is preferable for a cap tread.

### Example 18: Production of Vulcanized Rubber Composition

In the same manner as in Example 16 except that SBR #1712 (manufactured by JSR) is used instead of styrene-butadiene copolymer rubber SBR #1500 (manufactured by JSR), the amount of process oil to be used is changed to 21 part by weight, and the timing of charging zinc oxide is changed to step 2, a rubber composition is obtained. The obtained rubber composition is heated at 160° C. to give a vulcanized rubber composition. Such vulcanized rubber composition is preferable for a cap tread.

### Production Example 4: Production of Compound (1-2)

### (1) Production of Intermediate (Compound (c))

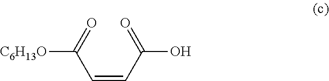

Under a nitrogen atmosphere, maleic anhydride (20.0 g, 203 mmol) and 1-hexanol (20.84 g, 203 mmol) were charged in a reactor, and the mixture was stirred at 90° C. for 2 hr. A mixture containing the obtained compound (c) was used without purification for the next reaction.

¹H NMR (400 MHz DMSO-d6) δ 0.86 (3H, t, J=6.8 Hz), 1.23-1.34 (6H, m), 1.54-1.61 (2H, m), 4.07 (2H, t, J=6.4 Hz), 6.33 (2H, d, J=12 Hz), 12.93 (1H, brs).

### (2) Production of Object Compound (Compound (1-2))

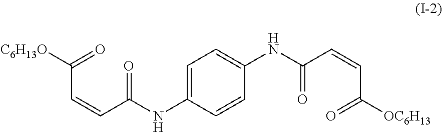

A mixture (40.79 g) containing compound (c) and dichloromethane (200 mL) were charged in a reactor at room temperature. Then, N-methylmorpholine (24.4 mL, 204 mmol) and isobutyl chloroformate (19.9 mL, 204 mmol) were charged in the reactor at 00° C., and the obtained mixture was stirred at 0° C. for 1 hr. Furthermore, a solution of benzene-1,4-diamine (11.0 g, 108.07 mmol) in dichloromethane (200 mL) was charged in the reactor at 0° C., and the obtained mixture was heated to room temperature and stirred at said temperature for 14 hr. Thereafter, the obtained mixture was concentrated, and the obtained concentrate was dissolved in ethyl acetate, and the obtained ethyl acetate solution was washed with 1N hydrochloric acid, then saturated aqueous NaHCO₃ solution, and then brine, and the obtained ethyl acetate layer was dried over anhydrous sodium sulfate. The dried ethyl acetate layer was concentrated to give object compound (I-2) (28.0 g, 59.3 mmol, yield 58%) as a yellow solid.

¹H NMR (400 MHz DMSO-d6) δ 0.83 (6H, t, J=6.8 Hz), 1.16-1.32 (12H, m), 1.53-1.58 (4H, m), 4.04 (4H, t, J=7.2 Hz), 6.33 (2H, d, J=12 Hz), 6.51 (2H, t, J=12 Hz), 7.58 (4H, s), 10.23 (2H, s).

### Example 19: Production of Vulcanized Rubber Composition

### <Step 1>

Using Banbury mixer (600 ml Labo Plastomill manufactured by Toyo Seiki Ltd.), natural rubber (RSS #1) (100 parts by weight), ISAF (trade name “Asahi #80” manufactured by Asahi Carbon Co., Ltd.) (45 parts by weight), stearic acid (3 parts by weight), zinc oxide (5 parts by weight), an anti-aging agent (N-phenyl-N′-1,3-dimethylbutyl-p-phenylenediamine, trade name “Antigen (registered trade mark) 6C” manufactured by Sumitomo Chemical Company, Limited) (1 part by weight) and compound (I-2) (1 part by weight) obtained in the above-mentioned Production Example 4 were kneaded to give a rubber composition. This step was performed by kneading at a mixer rotation speed of 50 rpm for 5 min after adding various components, with apparatus temperature at the start of kneading of 120° C., and the temperature of the rubber composition at discharge was 160-170° C.

### <Step 2>

The rubber composition obtained in step 1, a vulcanization accelerator (N-cyclohexyl-2-benzothiazolylsulfenamide) (1 part by weight) and powdered sulfur (2 parts by weight) were kneaded in an open roll at 60-80° C. to give a rubber composition.

### <Vulcanization>

The rubber composition obtained in step 2 was heated at 145° C. to give a vulcanized rubber composition. Vulcanization was performed for a time period of the 90% vulcanizing time (tc(90)) obtained by rheometer measurement according to JIS K 6300-2 plus 5 min.

### Reference Example 2: Production of Vulcanized Rubber Composition

In the same manner as in Example 19 except that compound (I-2) was not used, a vulcanized rubber composition was obtained.

### Experimental Example 2: Measurement of Loss Factor Lowering Effect

In the same manner as in Experimental Example 1, the loss factor (tan δ) of the vulcanized rubber compositions obtained in Example 19 and Reference Example 2 at 60° C. was determined, and a loss factor lowering effect (%) of the vulcanized rubber composition obtained in Example 19 was calculated by the following formula (2):

loss factor lowering effect (%)=100×(loss factor of Reference Example 2−loss factor of Example 19)/(loss factor of Reference Example 2) (2)

The loss factor lowering effect (%) was 10%.

### Example 20: Production of Vulcanized Tire

By covering a steel cord, which is subjected to a brass plating treatment, with the rubber composition obtained in Example 19, a belt is obtained. Using the obtained belt, a green tire is formed according to a general production method, and the obtained green tire is heated and pressurized in a vulcanizer to give a vulcanized tire.

### Example 21: Production of Vulcanized Tire

The rubber composition obtained in Example 19 is extrusion processed to give a tread member. Using the obtained tread member, a green tire is formed according to a general production method, and the obtained green tire is heated and pressurized in a vulcanizer to give a vulcanized tire.

### Example 22: Production of Vulcanized Tire

The rubber composition obtained in Example 19 is extrusion processed to prepare a rubber composition having a shape corresponding to the carcass shape, which is adhered to the top and bottom of a polyester carcass fiber cord to give a carcass. Using the obtained carcass, a green tire is formed according to a general production method, and the obtained green tire is heated and pressurized in a vulcanizer to give a vulcanized tire.

### Example 23: Production of Rubber Composition

In the same manner as in Example 19 except that N-(cyclohexylthio)-phthalimide (CTP) (0.2 parts by weight) is further added, a rubber composition is obtained.

### Example 24: Production of Rubber Composition

In the same manner as in Example 19 except that o,o′-dibenzamidodiphenyl disulfide (“NOCTIZER SS” manufactured by OUCHI SHINKO CHEMICAL INDUSTRIAL CO., LTD.) (0.2 parts by weight) is further added in step 1, a rubber composition is obtained.

### Example 25: Production of Vulcanized Rubber Composition

### <Step 1>

Using Banbury mixer (600 ml Labo Plastomill manufactured by Toyo Seiki Ltd.), styrene-butadiene copolymer rubber SBR #1502 (manufactured by Sumitomo Chemical Company, Limited) (100 parts by weight), ISAF-HM (trade name “Asahi #80” manufactured by Asahi Carbon Co., Ltd.) (45 parts by weight), stearic acid (2 parts by weight), zinc oxide (3 parts by weight), compound (I-2) (1 part by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (1 part by weight) and wax (“OZOACE-0355” manufactured by Nippon Seiro Co., Ltd.) (2 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N-cyclohexyl-2-benzothiazolylsulfenamide (CBS)) (3 parts by weight) and powdered sulfur (2 parts by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for a cap tread.

### Example 26: Production of Rubber Composition

### <Step 1>

Using Banbury mixer (600 ml Labo Plastomill manufactured by Toyo Seiki Ltd.), styrene-butadiene copolymer rubber SBR #1502 (manufactured by Sumitomo Chemical Company, Limited) (100 parts by weight), ISAF-HM (trade name “Asahi #80” manufactured by Asahi Carbon Co., Ltd.) (35 parts by weight), stearic acid (2 parts by weight), zinc oxide (3 parts by weight), compound (I-2) (1 part by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (1 part by weight) and wax (“OZOACE-0355” manufactured by Nippon Seiro Co., Ltd.) (2 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N-cyclohexyl-2-benzothiazolylsulfenamide (CBS)) (2 parts by weight), a vulcanization accelerator (diphenylguanidine (DPG)) (0.5 parts by weight), a vulcanization accelerator (dibenzothiazyl disulfide (MBTS)) (0.8 parts by weight) and powdered sulfur (1 part by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for an under tread.

### Example 27: Production of Vulcanized Rubber Composition

### <Step 1>

Using Banbury mixer (600 ml Labo Plastomill manufactured by Toyo Seiki Ltd.), natural rubber (RSS #1) (100 parts by weight), HAF (trade name “Asahi #70” manufactured by Asahi Carbon Co., Ltd.) (45 parts by weight), stearic acid (3 parts by weight), zinc oxide (5 parts by weight), compound (I-2) (1 part by weight), hydrated silica (“Nipsil (registered trade mark) AQ” manufactured by Tosoh Silica Corporation) (10 parts by weight), an anti-aging agent (“Antioxidant FR” manufactured by MATSUBARA SANGYO) (2 parts by weight), resorcinol (2 parts by weight) and cobalt naphthenate (2 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N,N-dicyclohexyl-2-benzothiazolylsulfenamide (DCBS)) (1 part by weight), insoluble sulfur (6 parts by weight) and methoxylated methylolmelamine resin (“SUMIKANOL 507AP” manufactured by Sumitomo Chemical Company, Limited) (3 parts by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for a belt.

### Example 28: Production of Vulcanized Rubber Composition

### <Step 1>

Using Banbury mixer (600 ml Labo Plastomill manufactured by Toyo Seiki Ltd.), halogenated butyl rubber (“Br-IIR 2255” manufactured by Exxon Mobil Corporation) (100 parts by weight), GPF (60 parts by weight), stearic acid (1 part by weight), zinc oxide (3 parts by weight), compound (I-2) (1 part by weight) and process oil (“Diana process PS32” manufactured by Idemitsu Kosan Co., Ltd.) (10 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, an anti-aging agent (reaction product of aniline and acetone (TMDQ)) (1 part by weight), a vulcanization accelerator (dibenzothiazyl disulfide (MBTS)) (1 part by weight) and powdered sulfur (2 parts by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for an inner liner.

### Example 29: Production of Vulcanized Rubber Composition

### <Step 1>

Using Banbury mixer (600 ml Labo Plastomill manufactured by Toyo Seiki Ltd.), natural rubber (RSS #3) (40 parts by weight), polybutadiene rubber (“BR150B” manufactured by Ube Industries, Ltd.) (60 parts by weight), FEF (50 parts by weight), stearic acid (2.5 parts by weight), zinc oxide (3 parts by weight), compound (I-2) (1 part by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (2 parts by weight), aromatic oil (“NC-140” manufactured by Cosmo Oil Co., Ltd.) (10 parts by weight) and wax (“SANNOC (registered trade mark) wax” manufactured by OUCHI SHINKO CHEMICAL INDUSTRIAL CO., LTD.) (2 parts by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N-tert-butyl-2-benzothiazolylsulfenamide (BBS)) (0.75 parts by weight) and powdered sulfur (1.5 parts by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for a side wall.

### Example 30: Production of Vulcanized Rubber Composition

### <Step 1>

Using Banbury mixer (600 ml Labo Plastomill manufactured by Toyo Seiki Ltd.), natural rubber (TSR20) (70 parts by weight), styrene-butadiene copolymer rubber SBR #1502 (manufactured by Sumitomo Chemical Company, Limited) (30 parts by weight), N339 (manufactured by Mitsubishi Chemical Corporation) (60 parts by weight), stearic acid (2 parts by weight), zinc oxide (5 parts by weight), process oil (“Diana process PS32” manufactured by Idemitsu Kosan Co., Ltd.) (7 parts by weight) and compound (I-2) (1 part by weight) are kneaded to give a rubber composition. This step is performed by kneading at a mixer rotation speed of 50 rpm for 5 min after adding respective components, and the temperature of the rubber composition at that time is 160-175° C.

### <Step 2>

The rubber composition obtained in step 1, a vulcanization accelerator (N-tert-butyl-2-benzothiazolylsulfenamide (BBS)) (1 part by weight), powdered sulfur (3 parts by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (1 part by weight) and an anti-aging agent (reaction product of aniline and acetone (TMDQ)) (1 part by weight) are kneaded in an open roll at 60-80° C. to give a rubber composition.

### <Vulcanization>

The rubber composition obtained in step 2 is heated at 145° C. to give a vulcanized rubber composition. Such vulcanized rubber composition is preferable for a carcass.

### Example 31: Production of Vulcanized Rubber Composition

### <Step 1>

Using Banbury mixer (600 ml Labo Plastomill manufactured by Toyo Seiki Ltd.), styrene-butadiene copolymer rubber SBR #1500 (manufactured by JSR) (100 parts by weight), silica (trade name: “Ultrasil (registered trade mark) VN3-G” manufactured by Degussa) (78.4 parts by weight), carbon black (trade name “N-339” manufactured by Mitsubishi Chemical Corporation) (6.4 parts by weight), a silane. coupling agent (bis(3-triethoxysilylpropyl)tetrasulfide, trade name “Si-69” manufactured by Degussa) (6.4 parts by weight), process oil (trade name “NC-140” manufactured by Cosmo Oil Co., Ltd.) (47.6 parts by weight), an anti-aging agent (N-phenyl-N′-1,3-dimethylbutyl-p-phenylenediamine (6PPD), trade name “Antigen (registered trade mark) 6C” manufactured by Sumitomo Chemical Company, Limited) (1.5 parts by weight), zinc oxide (2 parts by weight), stearic acid (2 parts by weight), and compound (I-2) (3 parts by weight) are kneaded to give a rubber composition. This step is performed within a temperature range of 70-120° C. by kneading at a mixer rotation speed of 80 rpm for 5 min after adding respective components, and successively kneading at a mixer rotation speed of 100 rpm for 5 min.

### <Step 2>

The rubber composition obtained in step 1, a vulcanization accelerator (N-cyclohexyl-2-benzothiazolylsulfenamide (CBS)) (1 part by weight), a vulcanization accelerator (diphenylguanidine (DPG)) (1 part by weight), wax (trade name “SANNOC (registered trade mark) N”, manufactured by OUCHI SHINKO CHEMICAL INDUSTRIAL CO., LTD.) (1.5 parts by weight) and powdered sulfur (1.4 parts by weight) are kneaded in an open roll at 30-80° C. to give a rubber composition.

### <Vulcanization>

The obtained rubber composition is heated at 160° C. to give a vulcanized rubber composition. Such vulcanized rubber composition is preferable for a cap tread.

### Example 32: Production of Vulcanized Rubber Composition

In the same manner as in Example 31 except that solution-polymerized SBR (“Asaprene (registered trade mark)” manufactured by Asahi Kasei Chemicals Corporation) is used instead of styrene-butadiene copolymer rubber SBR #1500 (manufactured by JSR), a rubber composition is obtained. The obtained rubber composition is heated at 160° C. to give a vulcanized rubber composition. Such vulcanized rubber composition is preferable for a cap tread.

### Example 33: Production of Vulcanized Rubber Composition

In the same manner as in Example 31 except that SBR #1712 (manufactured by JSR) is used instead of styrene-butadiene copolymer rubber SBR #1500 (manufactured by JSR), the amount of process oil to be used is changed to 21 part by weight, and the timing of charging zinc oxide is changed to step 2, a rubber composition is obtained. The obtained rubber composition is heated at 160° C. to give a vulcanized rubber composition. Such vulcanized rubber composition is preferable for a cap tread.

### INDUSTRIAL APPLICABILITY

According to the present invention, a loss factor (tan δ) of a vulcanized rubber composition can be lowered. The rubber composition and vulcanized rubber composition of the present invention are useful for the production of various products (e.g., vulcanized tire, tire member, vibration-proof rubber, conveyor belt rubber, engine mount rubber etc.).

This application is based on a patent application No. 2015-093469 filed in Japan, the contents of which are incorporated in full herein.

1. A rubber composition obtained by kneading a compound represented by the formula (I): 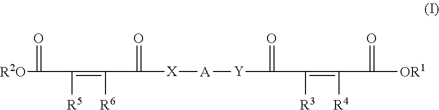
in the formula (I),
R¹ and R² are each independently a C₁₋₁₂ alkyl group optionally having one or more substituents or a C₃₋₁₀ cycloalkyl group optionally having one or more substituents,
R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents,
X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and
A is a divalent C₆₋₁₂ aromatic hydrocarbon group optionally having one or more substituents,
a rubber component and a filler.

2. The rubber composition according to claim 1, wherein R³, R⁴, R⁵ and R⁶ are each a hydrogen atom.

3. The rubber composition according to claim 1, wherein the compound represented by the formula (I) is a compound represented by the formula (II): 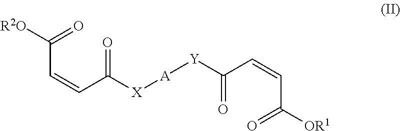
in the formula (II),
R¹ and R² are each independently a C₁₋₁₂ alkyl group optionally having one or more substituents or a C₃₋₁₀ cycloalkyl group optionally having one or more substituents,
X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and
A is a divalent C₆₋₁₂ aromatic hydrocarbon group optionally having one or more substituents.

4. The rubber composition according to claim 1, wherein R¹ and R² are each independently a linear C₁₋₆ alkyl group.

5. The rubber composition according to claim 1, wherein A is a phenylene group.

6. The rubber composition according to claim 1, wherein X and Y are each independently —NH— or —O—.

7. The rubber composition according to claim 1, wherein X and Y are each —NH—.

8. The rubber composition according to claim 1, wherein the rubber component comprises a diene rubber.

9. The rubber composition according to claim 1, wherein the filler comprises carbon black.

10. The rubber composition according to claim 1, which is obtained by further kneading a sulfur component.

11. A vulcanized rubber composition obtained by vulcanizing the rubber composition according to claim 10.

12. A vulcanized tire produced using the rubber composition according to claim 10.

13. A vulcanized tire comprising the vulcanized rubber composition according to claim 11.

14. A tire belt member comprising the vulcanized rubber composition according to claim 11 and a steel cord.

15. A tire carcass member comprising the vulcanized rubber composition according to claim 11 and a carcass fiber cord.

16. A tire member comprising the vulcanized rubber composition according to claim 11.

17. The tire member according to claim 16, which is a tire side wall member, a tire inner liner member, a tire cap tread member or a tire under tread member.

18. A loss factor lowering agent comprising a compound represented by the formula (I): 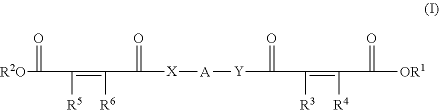
in the formula (I),
R¹ and R² are each independently a C₁₋₁₂ alkyl group optionally having one or more substituents or a C₃₋₁₀ cycloalkyl group optionally having one or more substituents,
R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents,
X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and
A is a divalent C₆₋₁₂ aromatic hydrocarbon group optionally having one or more substituents,
which is for a vulcanized rubber composition.

19. Use of a compound represented by the formula (I): 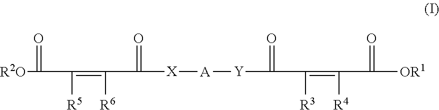
in the formula (I),
R¹ and R² are each independently a C₁₋₁₂ alkyl group optionally having one or more substituents or a C₃₋₁₀ cycloalkyl group optionally having one or more substituents,
R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents,
X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and
A is a divalent C₆₋₁₂ aromatic hydrocarbon group optionally having one or more substituents,
for lowering a loss factor of a vulcanized rubber composition.

20. A method of lowering a loss factor of a vulcanized rubber composition, comprising kneading a compound represented by the formula (I): 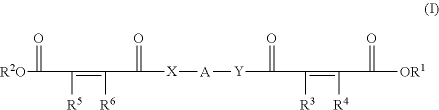
in the formula (I),
R¹ and R² are each independently a C₁₋₁₂ alkyl group optionally having one or more substituents or a C₃₋₁₀ cycloalkyl group optionally having one or more substituents,
R³, R⁴, R⁵ and R⁶ are each independently a hydrogen atom, a halogen atom, a hydroxy group, a C₁₋₆ alkoxy group optionally having one or more substituents, a C₁₋₆ alkyl group optionally having one or more substituents, a C₃₋₆ cycloalkyl group optionally having one or more substituents, or a C₆₋₁₄ aryl group optionally having one or more substituents, or R³ and R⁴ are bonded to form, together with the carbon atom bonded thereto, a C₃₋₁₀ cycloalkenediyl group optionally having one or more substituents, or R⁵ and R⁶ are bonded to form, together with the carbon atom bonded thereto, a C₃₋₁₀ cycloalkenediyl group optionally having one or more substituents,
X and Y are each independently —NR⁷—, —O—, —S—, or a C₁₋₁₂ alkanediyl group optionally having one or more substituents, R⁷ is a hydrogen atom or a C₁₋₁₂ alkyl group optionally having one or more substituents, and
A is a divalent C₆₋₁₂ aromatic hydrocarbon group optionally having one or more substituents,
a rubber component, a filler, and a sulfur component.
